(19) United States
(12) Patent Application Publication
(10) Pub. No.: US 2025/0295753 A1
(43) Pub. Date: Sep. 25, 2025

(54) Larygotracheitis Virus (LT) Vector Vaccines for use with Avian Respiratory Pathogens

(71) Applicant: EDGE ANIMAL HEALTH, INC., Boston, MA (US)

(72) Inventors: Kristi Mae MOORE, Shawnee, KS (US); Michelle Lynn HAVEN, Hampton, NJ (US); Richard E. PETERSON, Ijamsville, MD (US)

(73) Assignee: EDGE ANIMAL HEALTH, INC., Boston, MA (US)

(21) Appl. No.: 19/087,482

(22) Filed: Mar. 22, 2025

Related U.S. Application Data
(60) Provisional application No. 63/568,726, filed on Mar. 22, 2024.

(51) Int. Cl. A61K 39/17; A61K 39/00; A61K 39/215; A61P 31/14; C12N 7/00; C12N 15/86

(52) U.S. Cl. CPC A61K 39/17; A61K 39/215; A61P 31/14; C12N 7/00; C12N 15/86; A61K 2039/5254; A61K 2039/5256; A61K 2039/542; A61K 2039/544; A61K 2039/545; A61K 2039/552; C12N 2710/16034; C12N 2710/16043; C12N 2760/18134

(57) ABSTRACT

The present invention provides compositions and methods for immunizing a subject in need thereof again an avian pathogen. In one aspect, the invention provides a composition for immunizing an avian subject against a pathogen comprising: a) a recombinant laryngotracheitis virus vector (rLT); and b) a fusion gene of an avian virus (F) inserted into the recombinant laryngotracheitis virus vector (rLT/F); wherein the recombinant laryngotracheitis virus vector inserted with the fusion gene of the avian virus (rLT/F) is deoptimized to generate deoptimized rLT/F vaccine candidates; and wherein the deoptimized rLT/F vaccine candidates are administered to the subject in need thereof.

### CROSS-REFERENCE TO RELATED APPLICATIONS

This application claims the benefit of and priority to U.S. Provisional Patent Application 63/568,726, filed on Mar. 22, 2024, which is specifically incorporated by reference herein in its entirety.

### FIELD

The present invention relates to novel deoptimized recombinant laryngotracheitis virus carrying the fusion gene of an avian virus (rLT/F), and methods of their use in poultry vaccines. The invention also relates to methods of developing a deoptimized recombinant laryngotracheitis virus rLT vaccine vector carrying fusion genes of pathogenic avian viruses.

### BACKGROUND OF THE INVENTION

Infectious Laryngotracheitis Virus (ILTV) is a large (160 kb) double stranded DNA herpes virus that causes an acute respiratory disease in chickens.

For the vast majority of producers in the US, LT vaccinations are given to long lived chickens as part of the normal vaccination program while in broilers these vaccines are only given during outbreaks (Garcia et al., Diseases of Poultry, 14^{th} ed., pp. 189-209 (2013)). This is due to multiple factors including safety and efficacy issues for current vaccine offerings as well as the frequency of outbreaks.

Currently there are two types of attenuated vaccines used to control the virus. The chicken embryo origin (CEO) vaccine works well in the face of an outbreak but can revert to virulence quickly and is often associated with mild disease (vaccinal LT). However, the CEO vaccines stimulate a robust immunity which suppresses shedding of field virus in vaccinated flocks, which is a valuable feature used to quickly control outbreaks. The second type, the tissue culture origin (TCO) vaccine, is a milder vaccine and safer to use, but it does not induce a strong immune response and thus is often insufficient for controlling an outbreak of the disease. Both of the current attenuated LT vaccines are administered to chickens 3-weeks old or older, usually through drinking water or by spray administration.

In addition to live attenuated vaccines, vectored vaccines have been developed for controlling LT using fowlpox vectors or herpes virus of turkeys' (HVT) vectors. The vectored vaccines are safe and can provide somewhat better protection than TCO vaccines, but full immunity isn't developed very quickly and when it is, these vaccines do not suppress shedding of the field virus leaving a gap in protection (Garcia, 2013). These and other deficiencies in the current vaccines leaves room for the development of an improved product that is safe, efficacious and useful in controlling disease.

In an attempt to provide improved vaccines, Avian viruses have been used as vectors to in recombinant vaccines. For example, herpesvirus of turkeys is an example of a well-known vector recombinantly produced to include inserts that will elicit a protective response not only against Marek's Disease but also against infection with other infectious agents such as Newcastle disease virus (NDV). For a useful review, see Gimeno et al, “Efficacy of Various HVT Vaccines (Conventional and Recombinant) Against Marek's Disease in Broiler Chickens: Effect of Dose and Age of Vaccination,” 2016, Avian Diseases 60 (3): 662-8. Thus, it is an object of the current invention to provide compositions and methods for immunizing an avian against avian viruses with an improved vaccine vector.

Another object contemplated to be within the scope of the present invention is using ILT as a vector provide protection against infection with other avian pathogens following vaccination. In one embodiment, the other pathogen is Newcastle disease virus. In another embodiment of an ILT vectored vaccine according to the present invention, an insert from a different avian pathogen may be used to provide a safe and efficacious vaccine. In one embodiment, an insert from infectious bronchitis virus (IBV), infectious laryngotracheitis virus (ILTV) or avian influenza (AI) is useful in eliciting an immune response may be used. In one embodiment, an Avian Paramyxovirus insert may be used. In one embodiment, an Avian Herpesvirus (AVH) insert may be used.

### SUMMARY OF THE INVENTION

Accordingly, the present invention provides compositions and methods for immunizing a subject in need thereof again an avian pathogen. In one aspect, the invention provides a composition for immunizing an avian subject against a pathogen comprising: a) a recombinant laryngotracheitis virus vector (rLT); and b) a fusion gene of an avian virus (F) inserted into the recombinant laryngotracheitis virus vector (rLT/F); wherein the recombinant laryngotracheitis virus vector inserted with the fusion gene of the avian virus (rLT/F) is deoptimized to generate deoptimized rLT/F vaccine candidates; and wherein the deoptimized rLT/F vaccine candidates are administered to the subject in need thereof.

In one embodiment, the avian virus is selected from Newcastle disease virus (NDV), infectious bronchitis virus (IBV), infectious laryngotracheitis virus (ILTV) or avian influenza (AI). In a particular embodiment, the avian virus is Newcastle disease virus (NDV), and the composition has at least 99% sequence identity to SEQ ID NO:1.

In another embodiment, the deoptimized vaccine candidates comprise a deoptimized coding sequence having at least 99% sequence identity to SEQ ID NO:22-26. In yet another embodiment, the deoptimized rLT/F vaccine is administered to the subject to induce an immune response against the pathogen using a gel administration.

Another aspect of the invention provides a deoptimized rLT/F vaccine for immunizing an avian subject in need thereof against a pathogen comprising the deoptimized rLT/F composition as disclosed herein, wherein an effective immunizing dose of the deoptimized rLT/F vaccine is administered to the subject to induce an immune response against the pathogen. In one embodiment, the pathogen is selected from Newcastle disease virus (NDV), infectious bronchitis virus (IBV), infectious laryngotracheitis virus (ILTV) or avian influenza (AI). In another embodiment, the avian virus is selected from Newcastle disease virus (NDV), infectious bronchitis virus (IBV), infectious laryngotracheitis virus (ILTV) or avian influenza (AI). Another embodiment provides a deoptimized vaccine, wherein gene targets are fully deoptimized within the LT genome.

Yet another aspect of the invention provides a method of developing the deoptimized rLT/F vaccine according to claim 5 comprising the steps of: a) inserting the fusion gene of an avian virus (F) in a recombinant laryngotracheitis vector (rLT); b) evaluating insertion sites of the avian virus fusion gene in the recombinant laryngotracheitis vector; c) deoptimizing the recombinant laryngotracheitis vector carrying the fusion gene of the avian virus (rLT/F); and d) generating deoptimized rLT/F vaccine candidates; wherein the deoptimized rLT/F vaccine is administered into an avian subject in need thereof. In another embodiment, the avian virus is selected from Newcastle disease virus (NDV), infectious bronchitis virus (IBV), infectious laryngotracheitis virus (ILTV) or avian influenza (AI). Another embodiment provides a deoptimized vaccine, wherein gene targets are fully deoptimized within the LT genome. In another embodiment, the recombinant laryngotracheitis vector comprises the laryngotracheitis genome.

In one embodiment, wherein the rLT/F is deoptimized using deoptimization based on codon pair bias. In another embodiment gene editing technologies are used to generate deoptimized rLT/F.

In additional embodiments, the vaccine is administered intratracheally. by drinking water, spray, or by gel droplets or beads. In some embodiment, the vaccine is administered in multiple, lowering doses. In other embodiments, the vaccine is administered in combination with other vaccinations. And some other embodiments, the vaccine is administered in combination with an infectious bronchitis (IB) vaccine.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 is a schematic of the components were introduced by nucleofection into an LMH adapted LT strain and selected based on PCR positivity, antigen expression as well as GFP fluorescence.

FIG. 2 is illustrative of the deoptimized US5 coding sequence insertion strategy.

FIG. 3 illustrative of the deoptimization of the sequences which are deoptimized at 12.5%, 25%, 50%, 75% or 95%.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways. In order to more fully appreciate the instant invention, the following definitions are provided.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

As used herein, a “vaccine” is a composition that is suitable for application to an animal (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a clinical disease arising from an infection with a wild-type micro-organism, i.e., strong enough for aiding in the prevention of the clinical disease, and/or preventing, ameliorating or curing the clinical disease.

As used herein, the term “avian” includes chicken, turkeys, ducks, game birds, including but not limited to, quail, pheasants, and geese, and ratites including but not limited to ostrich and emu. The term “poultry” denotes birds of the order Galliformes such as, for example, ordinary domestic fowl.

As used herein, the term “aids in the protection” does not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, an “adjuvant” is a substance that is able to favor or amplify the cascade of immunological events, ultimately leading to a better immunological response, i.e., the integrated bodily response to an antigen. An adjuvant is in general not required for the immunological response to occur, but favors or amplifies this response.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient.

As used herein, “systemic administration” is administration into the circulatory system of the body (comprising the cardiovascular and lymphatic system), thus affecting the body as a whole rather than a specific locus such as the gastro-intestinal tract (via e.g., oral or rectal administration) and the respiratory system (via e.g., intranasal administration). Systemic administration can be performed e.g., by administering into muscle tissue (intramuscular), into the dermis (intradermal or transdermal), underneath the skin (subcutaneous), underneath the mucosa (submucosal), in the veins (intravenous) etc.

As used herein the term “parenteral administration” includes subcutaneous injections, submucosal injections, intravenous injections, intramuscular injections, intradermal injections, and infusion.

The term “approximately” is used interchangeably with the term “about” and signifies that a value is within twenty-five percent of the indicated value i.e., a peptide containing “approximately” 100 amino acid residues can contain between 75 and 125 amino acid residues.

As used herein, the term, “polypeptide” is used interchangeably with the terms “protein” and “peptide” and denotes a polymer comprising two or more amino acids connected by peptide bonds. The term “polypeptide” as used herein includes a significant fragment or segment, and encompasses a stretch of amino acid residues of at least about 8 amino acids, generally at least about 12 amino acids, typically at least about 16 amino acids, preferably at least about 20 amino acids, and, in particularly preferred embodiments, at least about 30 or more amino acids, e.g., 35, 40, 45, 50, etc. Such fragments may have ends which begin and/or end at virtually all positions, e.g., beginning at residues 1, 2, 3, etc., and ending at, e.g., 155, 154, 153, etc., in all practical combinations. Optionally, a polypeptide may lack certain amino acid residues that are encoded by a gene or by an mRNA. For example, a gene or mRNA molecule may encode a sequence of amino acid residues on the N-terminus of a polypeptide (i.e., a signal sequence) that is cleaved from, and therefore, may not be part of the final protein.

As used herein the term “antigenic fragment” in regard to a particular protein (e.g., a protein antigen) is a fragment of that protein (including large fragments that are missing as little as a single amino acid from the full-length protein) that is antigenic, i.e., capable of specifically interacting with an antigen recognition molecule of the immune system, such as an immunoglobulin (antibody) or T cell antigen receptor. For example, an antigenic fragment of an NDV fusion protein, is a fragment of that fusion protein that is antigenic. Preferably, an antigenic fragment of the present invention is immunodominant for antibody and/or T cell receptor recognition.

As used herein an amino acid sequence is 100% “homologous” to a second amino acid sequence if the two amino acid sequences are identical, and/or differ only by neutral or conservative substitutions as defined below.

Accordingly, an amino acid sequence is about 80% “homologous” to a second amino acid sequence if about 80% of the two amino acid sequences are identical, and/or differ only by neutral or conservative substitutions. Functionally equivalent amino acid residues often can be substituted for residues within the sequence resulting in a conservative amino acid substitution. Such alterations define the term “a conservative substitution” as used herein. For example, one or more amino acid residues within the sequence can be substituted by another amino acid of a similar polarity, which acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred conservative substitutions are: Lys for Arg and vice versa such that a positive charge may be maintained; Glu for Asp and vice versa such that a negative charge may be maintained; Ser for Thr such that a free-OH can be maintained; and Gln for Asn such that a free NH2 can be maintained. The amino acids also can be placed in the following similarity groups: (1) proline, alanine, glycine, serine, and threonine; (2) glutamine, asparagine, glutamic acid, and aspartic acid; (3) histidine, lysine, and arginine; (4) cysteine; (5) valine, leucine, isoleucine, methionine; and (6) phenylalanine, tyrosine, and tryptophan. In a related embodiment, two highly homologous DNA sequences can be identified by their own homology, or the homology of the amino acids they encode. Such comparison of the sequences can be performed using standard software available in sequence data banks. In a particular embodiment two highly homologous DNA sequences encode amino acid sequences having about 80% identity, more preferably about 90% identity and even more preferably about 95% identity. More particularly, two highly homologous amino acid sequences have about 80% identity, even more preferably about 90% identity and even more preferably about 95% identity.

As used herein, protein and DNA sequence percent identity can be determined using software such as MacVector v9, commercially available from Accelrys (Burlington, Mass.) and the Clustal W algorithm with the alignment default parameters, and default parameters for identity. See, e.g., Thompson, et al., 1994. Nucleic Acids Res. 22:4673-4680. ClustalW is freely downloadable for Dos, Macintosh and Unix platforms from, e.g., EMBLI, the European Bioinformatics Institute. These and other available programs can also be used to determine sequence similarity using the same or analogous default parameters. As used herein the terms “polynucleotide”, or a “nucleic acid” or a “nucleic acid molecule” are used interchangeably and denote a molecule comprising nucleotides including, but is not limited to, RNA, cDNA, genomic DNA and even synthetic DNA sequences. The terms are also contemplated to encompass nucleic acid molecules that include any of the art-known base analogs of DNA and RNA.

A nucleic acid “coding sequence” or a “sequence encoding” a particular protein or peptide, is a nucleotide sequence which is transcribed and translated into a polypeptide in vitro or in vivo when placed under the control of appropriate regulatory elements.

The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., avian) DNA, and even synthetic DNA sequences. A transcription termination sequence can be located 3′ to the coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the term “transcription terminator sequence” is used interchangeably with the term “polyadenylation regulatory element” and is a sequence that is generally downstream from a DNA coding region and that may be required for the complete termination of the transcription of that DNA coding sequence.

As used herein an “expression cassette” is a recombinant nucleic acid that minimally comprises a promoter and a heterologous coding sequence operably linked to that promoter. In many such embodiments, the expression cassette further comprises a transcription terminator sequence.

A “heterologous nucleotide sequence” as used herein is a nucleotide sequence that is added to a nucleotide sequence of the present invention by recombinant methods to form a nucleic acid that is not naturally formed in nature. Heterologous nucleotide sequences can also encode fusion (e.g., chimeric) proteins. In addition, a heterologous nucleotide sequence can encode peptides and/or proteins that contain regulatory and/or structural properties. In other such embodiments, a heterologous nucleotide sequence can encode a protein or peptide that functions as a means of detecting the protein or peptide encoded by the nucleotide sequence of the present invention after the recombinant nucleic acid is expressed. In still another embodiment, the heterologous nucleotide sequence can function as a means of detecting a nucleotide sequence of the present invention. A heterologous nucleotide sequence can comprise non-coding sequences including restriction sites, regulatory sites, promoters and the like.

A “codon” as used herein is specific sequence of three adjacent nucleotide bases on a strand of DNA or RNA that provides genetic code information for a particular amino acid or a termination signal. Codons can be deoptimized, for example, by manipulating the nucleic acid sequence using molecular biology methods. Attenuated pathogens, such as an attenuated virus or bacterium, can be used in an immune composition to stimulate an immune response in a subject. For example, attenuated pathogens can be used in an attenuated vaccine to produce an immune response without causing the severe effects of the disease. Particular examples of attenuated vaccines include, but are not limited to, measles, mumps, rubella, polio, typhoid, yellow fever, and varicella vaccines.

An “attenuated pathogen” as used herein is a pathogen with a decreased or weakened ability to produce disease while retaining the ability to stimulate an immune response like that of the natural pathogen. In one example, a live pathogen is attenuated by deoptimizing one or more codons in one or more genes, such as an immunogenic surface antigen or a housekeeping gene. In another example, a pathogen is attenuated by selecting for avirulent variants under certain growth conditions (for example see Sabin and Boulger. J. Biol. Stand. 1:115-8; 1973; Sutter et al., 2003. Poliovirus vaccine—live, p. 651-705. In S. A. Plotkin and W. A. Orenstein (ed.), Vaccines, Fourth ed. W.B. Saunders Company, Philadelphia).

“Deoptimization of a codon” as used herein is to replace a preferred codon in a nucleic acid sequence with a synonymous codon (one that codes for the same amino acid) less frequently used (unpreferred) in the organism. Each organism has a particular codon usage bias for each amino acid, which can be determined from publicly available codon usage tables (for example see 25 Nakamura et al., Nucleic Acids Res. 28:292, 2000 and references cited therein; Sharp et al., Nucleic Acids Res. 16:8207-11, 1988; Chou and Zhang, AIDS Res. Hum. Retroviruses. December; 8 (12): 1967-76, 1992; West and Iglewski et al., Nucleic Acids Res. 16:9323-35, 1988, Rothberg and Wimmer, Nucleic Acids Res. 9:6221-9, 1981; Jenkins et al., J. Mol. Evol. 52:383-90, 2001; and Watterson, Mol. Biol. Evol. 9:666-77, 1992; all herein incorporated by reference). In addition, codon usage tables are available for several organisms on the internet at GenBank's website.

A “deoptimized pathogen” as used herein is a pathogen having a nucleic acid coding sequence with one or more deoptimized codons, which decrease the replicative fitness of the pathogen. Some examples refer to the isolated deoptimized nucleic acid sequence itself, independent of the pathogenic organism.

### II. Compositions

The compositions and methods provide herein a deoptimized recombinant laryngotracheitis virus vector carrying, in one embodiment, the fusion gene of Newcastle disease virus (rLT/F), and methods of their use in poultry vaccines. However, it is also contemplated to be within scope of the present invention that the rLT vector can also be used with inserts from other avian pathogens, useful in eliciting an immune response in a recipient, such as infectious bronchitis virus, Avian influenza virus, and the like.

### A. Infectious Laryngotracheitis (ILT)

Infectious laryngotracheitis (ILT) is an acute respiratory disease of chickens that causes significant economic losses to poultry industry worldwide (Bagust et al., 2000, Rev Sci Tech 19, 483-492; Bagust, 1986, Avian Pathol 15, 581-595). The causative pathogen, ILTV, is a member of the genus Iltovirus in the family Herpesviridae (Bagust et al., 2000, supra; Fuchs et al., 2007, Vet Res 38, 261-279). Currently, live attenuated vaccines are used to control ILT infections. However, the live-attenuated vaccines are not satisfactory since they can revert to virulence after bird-to-bird passage (Guy et al., 1991, Avian Dis 35, 348-355) and can induce latent infections (Hughes et al., 1991, Arch Virol 121, 213-218). Several alternative strategies have been used to develop improved ILTV vaccines (Mauricio et al., 2013, Avian Pathol 42, 195-205). One of the strategies has been the creation of ILTV deletion mutants for use as attenuated live-virus vaccines (Mauricio et al., 2013, supra). Two of the concerns of using gene deleted ILTV vaccine are the establishment of latency and the possibility that the gene-deleted vaccine virus could become virulent after recombination with different attenuated vaccine used in the same region (Sang-Won et al, 2012, Science 337, 188; Henderson et al., 1991, Am J Vet Res 52, 820-825). All studies conducted to date suggest that a virus-vectored ILTV vaccine will be most effective for prevention and control of ILT (Tong et al., 2001, Avian pathol 30, 143-148; Sun et al., 2008, Avian Dis 52, 111-117; Vagnozzi et al., 2012, Avian Pathol 41, 21-31). A vectored-vaccine will be safe and not lead to reversion to virulence or establishment of latency. However, current live virus vectored vaccines against ILT have limitations (Mauricio et al., 2013, supra; Vagnozzi et al. 2012, supra): (i) route of administration to largenumber of one-day old chicks, (ii) effective delivery of vaccine antigen to the mucosal surface, (iii) production cost, and (iv) incomplete protection. Therefore, there is a need to evaluate additional viral vectors to deliver ILTV antigens to chickens.

The ILTV gD gene appears to encode a glycoprotein of 434 amino acids in length having a molecular weight of 48,477 daltons, although others have suggested that a downstream start codon, which leads to an ILTV gD protein comprising only 377 amino acid residues, is the actual start codon [Wild et al., Virus Genes 12:104-116 (1996)]. The ILTV gI gene encodes a glycoprotein of 362 amino acids in length having a molecular weight of 39,753 daltons [U.S. Pat. No. 6,875,856, hereby incorporated by reference]. Nucleic acids encoding natural and/or laboratory derived variants of the ILTV gD and ILTV gI may be substituted for those presently exemplified

### B. Newcastle Disease Virus

Newcastle disease is a highly contagious viral disease affecting all species of birds. The disease can vary from an asymptomatic infection to a highly fatal disease, depending on the virus strain and the host species. Newcastle disease has a worldwide distribution and is a major threat to the poultry industries of all countries. Based on the severity of the disease produced in chickens, Newcastle disease virus (NDV) strains are grouped into three mainpathotypes: lentogenic (strains that do not usually cause disease in adult chickens), mesogenic (strains of intermediate virulence) and velogenic (strains that cause high mortality). NDV is a member of the genus Rubulavirus in the family Paramyxoviridae. The genome of NDV is a non-segmented, single-stranded, negative-sense RNA of 15186 nucleotides (Krishnamurthy & Samal, 1998, J Gen Virol 79, 2419-2424; Phillips et al., 1998, Arch Virol 143, 1993-2002; de Leeuw and Peeters, 1999, J Gen Virol 80, 131-136). The genomic RNA contains six genes that encode the following proteins in the order of: the nucleocapsid protein (NP), phosphoprotein (P), matrix protein (M), fusion protein (F), haemagglutinin-neuraminidase (HN) and large polymerase protein (L). Two additional proteins, V and W, of unknown function are produced by RNA editing during P gene transcription (Steward et al., 1993, J Gen Virol 74, 2539-2547).

Three proteins, i.e. NP, P and L proteins, constitute the nucleocapsid. The genomic RNA is tightly bound by the NP protein and together with the P and L proteins form the functional nucleocapsid within which resides the viral transcriptive and replicative activities. The F and HN proteins form the external envelope spikes, where the HN glycoprotein is responsible for attachment of the virus to host cell receptors and the F glycoprotein mediates fusion of the viral envelope with the host cell plasma membrane thereby enabling penetration of the viral genome into the cytoplasm of the host cell. The HN and F proteins are the main targets for the immune response. The M protein forms the inner layer of the virion.

NDV follows the general scheme of transcription and replication of other non-segmented negative-strand RNA viruses. The polymerase enters the genome at a promoter in the 3′ extragenic leader region and proceeds along the entire length by a sequential stop-start mechanism during which the polymerase remains template bound and is guided by short consensus gene start (GS) and gene end (GE) signals. This generates a free leader RNA and six non-overlapping subgenomic mRNAs. The abundance of the various mRNAs decreases with increasing gene distance from the promoter. The genes are separated by short intergenic regions (1-47 nucleotides) which are not copied into the individual mRNAs. RNA replication occurs when the polymerase somehow switches to a read-through mode in which the transcription signals are ignored. This produces a complete encapsulated positive-sense replicativeintermediate which serves as the template for progeny genomes.

### C. Promoters and Polyadenylation Regulatory Elements

Many alternative promoters can be used to drive the expression of a heterologous gene encoding a protein antigen or antigenic fragment thereof in a deoptimized rLT/F of the present invention. Examples include the pseudorabies virus (PRV) gpX promoter [see, WO 87/04463], the Rous sarcoma virus LTR promoter, the SV40 early gene promoter, the ILTV gD promoter, the ILTV gI promoter [see e.g., U.S. Pat. No. 6,183,753 B1], the human cytomegalovirus immediate early 1 (hCMV IE1) gene promoter [U.S. Pat. Nos. 5,830,745; 5,980,906], and the chicken beta-actin gene promoter [EP 1 298 139B1].

The inclusion of a polyadenylation regulatory element downstream from a DNA coding region is oftentimes required to terminate the transcription of the coding DNA sequence. Accordingly, many genes comprise a polyadenylation regulatory element at the downstream end of their coding sequence. Many such regulatory elements have been identified and can be used in a deoptimized rLT/F of the present invention.

### D. ILT Recombinant-NDV-F Transgene Sequences

### 1. ILT Recombinant-NDV-F Transgene Insertion Sequence

Provided herein is the nucleic acid sequence for the ILT recombinant vector with the NDV-F transgene insertion having at least 90% sequence identity to:

| (SEQ ID NO: 1) | |
|---|---|
| 1 | atgcagcatc agagtactgc gctagtttcg agtatacttt tgctcttgag cctgcaaagc |
| 61 | cttgcgtttg aatttttctg tgatccgcca cacgtttttc gagggcagct cggtgacccc |
| 121 | attctattgc aatgcttcag cgacagacct ctaacccacg aagaatctgt aaaagtagaa |
| 181 | gtaattcgac acccagccag cttagttgaa actgcgctaa gcgcctacgg gatcccccct |
| 241 | tcgctagatc catggagagc tactccaaga actctctaca catatgatgc cgctactgat |
| 301 | tcaatcaagg acctaggata cattggtgaa gatggaatta acccaccata tttggacgac |
| 361 | tgtcgttcag gttttttcaa tgtctctatc aagtctagca tgagatctca catggcgcgt |
| 421 | tatcagtgga ccgcaagtcg agggtctaca aaactaaata gctcttttat cgacgtcttt |
| 481 | ttggcaagac cacctacaac tgtccgcatc aaatcagaag aactgtacga agactcagat |
| 541 | aaggcttcgc acttaagtgt tgaagcgctt ggcgcttatc ctccatctgc tgcgctgggt |
| 601 | acatggatga tacataatgc atctcttgct gaaaaataca gtttagaaag aagagttctt |
| 661 | tatgcatcag gagagaatgg atcggtggat cagacatggg aactggaaat acgtggagaa |
| 721 | gccagccagc ccctcccttc caaaattcaa tttgtatatc gatggacccc tcctgaggac |
| 781 | tttgaaatgc tacgacctga aactcgcttg ttaaggttga ctcccagctg gattagcaag |
| 841 | ccccgcatca cggtacaatt cgtccctcct gcctatgccc tgtgtagagc agctaatatt |
| 901 | atagacggcc gaggatttat tgaatggatc gtagataata gaatttcgac gagcccacac |
| 961 | cagacctttg ttttggatga gcccgagggg aaaaatatcg ttacactaat ggacgtcata |
| 1021 | aaactaccac cggaggatac atttcaatct gcctctaatt acgtgtgcgt cataagaggc |
| 1081 | tatgaacatg catacagata tctcaacgcc tccttaatga tagataatct gccaatgcgg |
| 1141 | caaggattcc ccgcagtcgc tgcgattttt attataatta gtatcgcttt tgtgggtggg |
| 1201 | ttactagttg cttgcttggg cgcatggtgc tggaagacaa cataaacgct catttaataa |
| 1261 | atgacattac aaacgtgcac taccaccgga gttattaata gtaatcaatt acggggtcat |
| 1321 | tagttcatag cccatatatg gagttccgcg ttacataact tacggtaaat ggcccgcctg |
| 1381 | gctgaccgcc caacgacccc cgcccattga cgtcaataat gacgtatgtt cccatagtaa |
| 1441 | cgccaatagg gactttccat tgacgtcaat gggtggagta tttacggtaa actgcccact |
| 1501 | tggcagtaca tcaagtgtat catatgccaa gtacgccccc tattgacgtc aatgacggta |
| 1561 | aatggatgca gtattttgtg cagcgatggg ggcggggcgg ggcgaggcgg agaggtgcgg |
| 1621 | cggcagccaa tcagagcggc gcgctccgaa agtttccttt tatggcgagg cggcggcggc |
| 1681 | ggcggcccta taaaaagcga agcgcgcggc gggcgggagt cgctgcgcgc tgccttcgcc |
| 1741 | ccgtgccccg ctccgccgcc gcctcgcgcc gcccgccccg gctctgactg accgcgtacc |
| 1801 | cactgcttac tggcttatcg aaattaatac gactcactat aggggatccg ccaccatggg |
| 1861 | cagcagacct agcaccaaaa atcccgctcc aatgatgctg acaatcagag tggctctggt |
| 1921 | gctgagctgc atttgcccag ctaacagcat tgacggaaga cccctggctg ccgctggcat |
| 1981 | cgtggtgacc ggcgacaaag ccgtgaatat ctataccagc agccagacag gcagcatcat |
| 2041 | tgtgaagctg ctgccaaatc tgcctaaaga taaagaggcc tgtgccaaag ctcctctgga |
| 2101 | cgcttacaac agaacactga caaccctgct gaccccactg ggagatagca tcagaagaat |
| 2161 | tcaggaaagc gtgacaacca gcggaggcgg aagacaaggc agactgatcg gagctattat |
| 2221 | cggcggagtg gccctgggcg tggctaccgc tgcccagatt acagccgctg ccgctctgat |
| 2281 | ccaggccaaa caaaacgctg ccaacatcct gagactgaag gaaagcatcg ccgctaccaa |
| 2341 | tgaggctgtg catgaggtga cagacggcct gagccagctg gccgtggctg tgggcaaaat |
| 2401 | gcagcaattc gtgaatgacc agttcaacaa aacagctcag gagctggact gcatcaagat |
| 2461 | cgcccaacag gtgggcgtgg aactgaatct gtacctgaca gaactgacca cagtgtttgg |
| 2521 | cccacaaatc accagcccag ctctgaacaa gctgaccatc caggccctgt acaacctggc |
| 2581 | tggcggaaat atggactacc tgctgaccaa gctgggcgtg ggcaataacc agctgagcag |
| 2641 | cctgattgga agcggactga ttaccggcaa tccaatcctg tatgacagcc agacccagct |
| 2701 | gctgggcatc caggtgacac tgcctagcgt gggcaacctg aataacatga gagccaccta |
| 2761 | tctggaaaca ctgagcgtga gcaccacaag aggctttgct agcgctctgg tgcctaaggt |
| 2821 | ggtgacccaa gtgggaagcg tgatcgagga actggacaca agctactgca tcgaaaccga |
| 2881 | cctggatctg tactgcacca gaattgtgac attccccatg agcccaggaa tttacagctg |
| 2941 | cctgagcgga aataccagcg cttgcatgta tagcaaaacc gaaggagctc tgaccacacc |
| 3001 | ttacatgacc atcaagggaa gcgtgatcgc taattgtaag atgacaacct gcagatgcgt |
| 3061 | gaatccccct ggcatcatta gccagaatta cggcgaggcc gtgagcctga ttgacaaaca |
| 3121 | gagctgcaac gtgctgagcc tgggcggaat taccctgaga ctgagcggag aattcgacgt |
| 3181 | gacatatcag aagaacatca gcattcaaga tagccaggtg atcattacag gcaacctgga |
| 3241 | tatcagcaca gaactgggca acgtgaataa cagcattagc aatgctctga acaagctgga |
| 3301 | ggaaagcaat agaaagctgg acaaggtgaa tgtgaagctg acaagcacaa gcgctctgat |
| 3361 | cacatacatc gtgctgacca ttatcagcct ggtgttcgga atcctgagcc tgattctggc |
| 3421 | ttgttatctg atgtataagc agaaggccca gcaaaaaaca ctgctgtggc tgggcaataa |
| 3481 | caccctggac caaatgagag ctacaaccaa gatgggctcc ggagaaggaa gaggctccct |
| 3541 | gctgacatgc ggagacgtgg aagagaaccc aggccctatg ggcgtgatca agcccgacat |
| 3601 | gaagatcaag ctgcggatgg agggcgccgt gaacggccac aaattcgtga tcgagggcga |
| 3661 | cgggaaaggc aagccctttg agggtaagca gactatggac ctgaccgtga tcgagggcgc |
| 3721 | ccccctgccc ttcgcttatg acattctcac caccgtgttc gactacggta accgtgtctt |
| 3781 | cgccaagtac cccaaggaca tccctgacta cttcaagcag accttccccg agggctactc |
| 3841 | gtgggagcga agcatgacat acgaggacca gggaatctgt atcgctacaa acgacatcac |
| 3901 | catgatgaag ggtgtggacg actgcttcgt gtacaaaatc cgcttcgacg gggtcaactt |
| 3961 | ccctgctaat ggcccggtga tgcagcgcaa gaccctaaag tgggagccca gtaccgagaa |
| 4021 | gatgtacgtg cgggacggcg tactgaaggg cgatgttaat atggcactgc tcttggaggg |
| 4081 | aggcggccac taccgctgcg acttcaagac cacctacaaa gccaagaagg tggtgcagct |
| 4141 | tcccgactac cacttcgtgg accaccgcat cgagatcgtg agccacgaca aggactacaa |
| 4201 | caaagtcaag ctgtacgagc acgccgaagc ccacagcgga ctaccccgcc aggccggcta |
| 4261 | acccggagaa ggaagaggct ccctgctgac atgcggagac gtggcacgtg ctacgagatt |
| 4321 | tcgattccac cgccgccttc tatgaaaggt tgggcttcgg aatcgttttc cgggacgccg |
| 4381 | gctggatgat cctccagcgc ggggatctca tgctggagtt cttcgcccac cccaacttgt |
| 4441 | ttattgcagc ttataatggt tacaaataaa gcaatagcat cacaaatttc acaaataaag |
| 4501 | catttttttc actgcattct agttgtggtt tgtccaaact catcaatgta tcttagagat |
| 4561 | cgagtgccgc atcatcggaa accttgcact ggcaggtagt ctcaagcaag cctaaacgat |
| 4621 | attatagatt tcaatatttc taccagatcc gtccgatatt cccaaatgat tggagaaatt |
| 4681 | tcttccttag tcatcttcca cagatcttcg attttgcgcc caagctcaaa atttccagta |
| 4741 | atttcagaaa cctcactggc cgcgtatgcc tcctcgtaca agtaaatgca gacgagagcc |
| 4801 | gcggcggttc gctttgcagt gttgaccaaa atttcttgtg gaattttagt tcgaaggcac |
| 4861 | gctgcggtac ttacatcttt cattaaacgg gtttttgccg catcgaggag taccgctgtt |
| 4921 | tctgggatta acgggatact ctctggtgga tacgaacatc tcggtttggg ttgcactttg |
| 4981 | ggattcaaac aacagctgac tagtgcatct tgcaaaacat agtgcgtggc cagcaaagtt |
| 5041 | ttcaatatat tgagaacaac ttccgacaat tcttgtaatt gtagagtttc ggggctctgg |
| 5101 | atttttttga gcccgctttt ttcaatatat ctttcacaga caactgtaat ttcgtcaaag |
| 5161 | tgacaaggat agtgcccgag cataagtaaa taattaaaca gagtataagg aatcactgag |
| 5221 | cttcgcccga atgatgcttt gctgaggcat cgttggccag ggtatggcag cgtgaccaga |
| 5281 | atgtcagcct cgtcgcgcaa tgtacttttc agttttgctc catcagtctc ccatgaaagt |
| 5341 | ccgatagttg cgagaagtcc atgtcgtgaa atcgtggccg catccacgat cattaacatc |
| 5401 | ttccagagtc gctttaatac tttccaacat tcccagaaat tttctacatc ttcgtcgaac |
| 5461 | atttggtcgg ggtaaatgtg tttcacctgc acatgatctg agaggatagt ttttttctta |
| 5521 | cacccacgca gggctttttt ctcatttcct actgcgaccg tactaatgat tttacctccc |
| 5581 | gtaataataa taggagtaaa aatctgtgat tgttcatgaa tcggttctct tgttagggag |
| 5641 | ttcaagaata aatcttctaa atcttcttct atgaacttcg gcagagtagt aatatgtgcc |
| 5701 | tgcgttagca ccagcgcact actatctact tgatacctaa atcctgtcgt cgtattgatt |
| 5761 | agatccgggg ttctaaataa cctcagtact ttattcctca gagagacttc cccaactcgt |
| 5821 | atttctagcg tttcggaaac ctgcaggccg acaaatattt tgcacaagat cggtcgtctg |
| 5881 | atctccacgg taaaattatc aatcaattca tcatgtggag attctgaaat ttcaccaaga |
| 5941 | tgagaagtaa aaatgtattc ttcctcctgg cggtcaaaag gcatcccatt caaacgattc |
| 6001 | agttcagtgt taataatcgc acacatggta gggtgtttga tcctaaaacc gaatttggta |
| 6061 | atatttttta tcggcccgcg agtagagatc gagtagatgc atcctccata gataatgtag |
| 6121 | agtcgtccgc tgtggtgatc tatgtagcgt gcaggagact ttgatccgcg cttggagact |
| 6181 | tcttgaaaag ccacttccat ttcagtcctt ctccaaagag cttgcgcgtc aagtgacggt |
| 6241 | accgctattg gtgctttaaa agtatccgcc ctttatactg ctccaattct atcgtagcat |
| 6301 | aaagttttga cgatcataca gcgcctgcgt cttggcacca cctacaacca aacctgagct |
| 6361 | ctccttcaac atgacaactg gaaacgaagc caattcaatc cgcctctctt cagtacaagt |
| 6421 | accgggaaat tgtgagattg acaagctgtt ggctggattt gactacgaaa aagaacgtga |
| 6481 | tggtgactat tcgacactga acgaaggatc catatttcta aacgatcgcc ttatagcttc |
| 6541 | gggaagtgaa gatacatctg atctacaata tacgcgaatg aggaaacaaa ctttttatcc |
| 6601 | tagtttagcc cttcttttaa aaatactctg ttgtctgcct ttcttttggt acgggaattg |
| 6661 | cacgcgagag aaatatttat ttgtaaacgc tcttattatc actgccctct cctgtctaga |
| 6721 | aggactattg atttcctttt ttgtgtaccg aaacgtgaag gctgatcgtc ttcctttaaa |
| 6781 | aggtcctgaa aaactcattc aaatggtgtt atgtatgata acggctatat atggggcgat |
| 6841 | tatcttttca aggcaccttt ttgcggacga tgaccttgct atttcaatat ttgcgaaaaa |
| 6901 | ctggacagac acacaggaag ccttgagaat tggtcattgt tatctctctc cgtatttttc |
| 6961 | gatgtgggct gcctgtctgt actttataat tcttctatac gacgttattg acgtgacact |
| 7021 | tcctcttttg tgggcctgga caatattaag gacagcaata agcttctaaa tcgttttact |
| 7081 | gctacacgaa atcgcaaagt taggtggaag gatcgcgtta ctggcctcat aaaccccgcc |
| 7141 | ttgaagcctc acccccggta aagacacgat aaaggcagag actgcagtat tataacgggc |
| 7201 | tgacttcgta ggataaaggc aacctacaac tctgtgtgct caatatttag ccatgaatcc |
| 7261 | tgacaacggg atcccgcata acagtcatca tgatcgcgca gcattcccaa gatctgctgc |
| 7321 | ccctttcgta gcatctgggg aactgttagg aattcttcga gaaaattgcc atgcgcatct |
| 7381 | atatgaatgg ataagccgcg aaggggattg ttgctacaga cacagctttg atattctgct |
| 7441 | gggatcttat ttcaatacac taacgctcac caactttcta gaaaccggac tttcagttgc |
| 7501 | atgtatttgc gtgaaatttc cagagttacg ctatgcagac cgaggaataa tccagtttgt |
| 7561 | agtggctaat cccatgattg caagaagtga ttgtgaagta ccttctcggc catcatttac |
| 7621 | ctacatcagt aagagatggt ctaggacgac attatcctca tcccttgtga tttgtgcacc |
| 7681 | agctctgggc ttgctaagtg gcgagtcact tgacgggacc gaaatatctg agttttctag |
| 7741 | attacaggcg ttaaaccaac ttgcacgaaa cctcaaacta actctagact catttgaaag |
| 7801 | aggaacaata aatcatgtgc tgagaattct aatccgaaaa gc |

Nucleci acids 1290-1576 of SEQ ID NO: 1 encode the enhancer sequence. In particular embodiments, the enhancer is a CMV enhancer having at least 90% identity to:

(SEQ ID NO: 2)
AGTTATTAATAGTAATCAATTACGGGGTCATTAGTTCATAGCCCATATA
TGGAGTTCCGCGTTACATAACTTACGGTAAATGGCCCGCCTGGCTGACC
GCCCAACGACCCCCGCCCATTGACGTCAATAATGACGTATGTTCCCATA
GTAACGCCAATAGGGACTTTCCATTGACGTCAATGGGTGGAGTATTTAC
GGTAAACTGCCCACTTGGCAGTACATCAAGTGTATCATATGCCAAGTAC
GCCCCCTATTGACGTCAATGACGGTAAATGGATGCAGTATTT.

Nucleic acid sequence 1577-1807 of SEQ ID NO: 1 encode the promoter sequence. In particular embodiments, the promoter is a chicken beta actin promoter having at least 90% sequence identity to:

(SEQ ID NO: 3)
TGTGCAGCGATGGGGGCGGGGCGGGGCGAGGCGGAGAGGTGCGGCGGCA
GCCAATCAGAGCGGCGCGCTCCGAAAGTTTCCTTTTATGGCGAGGCGGC
GGCGGCGGCGGCCCTATAAAAAGCGAAGCGCGCGGCGGGCGGGAGTCGC
TGCGCGCTGCCTTCGCCCCGTGCCCCGCTCCGCCGCCGCCTCGCGCCGC
CCGCCCCGGCTCTGACTGACCGCGTACCCACTGCT.

The enhancer (SEQ ID NO:2) and promoter (SEQ ID NO:3) sequences of SEQ ID NO: 1 direct high-level expression of the ORF encoding the NDV-F antigen and GFP fluorescent marker for identification of recombinants.

Translation of the NDV-F gene that is fused to a 2A peptide sequence from a virus Thosea asigna (T2A), followed by the coding sequence for green fluorescent protein (GFP). When translated the T2A will direct a ribosomal skip that will result in 2 independent polypeptide sequences produced, the NDV-F protein and a GFP protein.

Nucleic acid sequences 1856-4261 are the open reading frame (ORF) sequences, In particular embodiments, the ORF is NDV-F t2a GFP having at least 90% sequence idendity to:

(SEQ ID NO: 4)
ATGGGCAGCAGACCTAGCACCAAAAATCCCGCTCCAATGATGCTGACAA
TCAGAGTGGCTCTGGTGCTGAGCTGCATTTGCCCAGCTAACAGCATTGA
CGGAAGACCCCTGGCTGCCGCTGGCATCGTGGTGACCGGCGACAAAGCC
GTGAATATCTATACCAGCAGCCAGACAGGCAGCATCATTGTGAAGCTGC
TGCCAAATCTGCCTAAAGATAAAGAGGCCTGTGCCAAAGCTCCTCTGGA
CGCTTACAACAGAACACTGACAACCCTGCTGACCCCACTGGGAGATAGC
ATCAGAAGAATTCAGGAAAGCGTGACAACCAGCGGAGGCGGAAGACAAG
GCAGACTGATCGGAGCTATTATCGGCGGAGTGGCCCTGGGCGTGGCTAC
CGCTGCCCAGATTACAGCCGCTGCCGCTCTGATCCAGGCCAAACAAAAC
GCTGCCAACATCCTGAGACTGAAGGAAAGCATCGCCGCTACCAATGAGG
CTGTGCATGAGGTGACAGACGGCCTGAGCCAGCTGGCCGTGGCTGTGGG
CAAAATGCAGCAATTCGTGAATGACCAGTTCAACAAAACAGCTCAGGAG
CTGGACTGCATCAAGATCGCCCAACAGGTGGGCGTGGAACTGAATCTGT
ACCTGACAGAACTGACCACAGTGTTTGGCCCACAAATCACCAGCCCAGC
TCTGAACAAGCTGACCATCCAGGCCCTGTACAACCTGGCTGGCGGAAAT
ATGGACTACCTGCTGACCAAGCTGGGCGTGGGCAATAACCAGCTGAGCA
GCCTGATTGGAAGCGGACTGATTACCGGCAATCCAATCCTGTATGACAG
CCAGACCCAGCTGCTGGGCATCCAGGTGACACTGCCTAGCGTGGGCAAC
CTGAATAACATGAGAGCCACCTATCTGGAAACACTGAGCGTGAGCACCA
CAAGAGGCTTTGCTAGCGCTCTGGTGCCTAAGGTGGTGACCCAAGTGGG
AAGCGTGATCGAGGAACTGGACACAAGCTACTGCATCGAAACCGACCTG
GATCTGTACTGCACCAGAATTGTGACATTCCCCATGAGCCCAGGAATTT
ACAGCTGCCTGAGCGGAAATACCAGCGCTTGCATGTATAGCAAAACCGA
AGGAGCTCTGACCACACCTTACATGACCATCAAGGGAAGCGTGATCGCT
AATTGTAAGATGACAACCTGCAGATGCGTGAATCCCCCTGGCATCATTA
GCCAGAATTACGGCGAGGCCGTGAGCCTGATTGACAAACAGAGCTGCAA
CGTGCTGAGCCTGGGCGGAATTACCCTGAGACTGAGCGGAGAATTCGAC
GTGACATATCAGAAGAACATCAGCATTCAAGATAGCCAGGTGATCATTA
CAGGCAACCTGGATATCAGCACAGAACTGGGCAACGTGAATAACAGCAT
TAGCAATGCTCTGAACAAGCTGGAGGAAAGCAATAGAAAGCTGGACAAG
GTGAATGTGAAGCTGACAAGCACAAGCGCTCTGATCACATACATCGTGC
TGACCATTATCAGCCTGGTGTTCGGAATCCTGAGCCTGATTCTGGCTTG
TTATCTGATGTATAAGCAGAAGGCCCAGCAAAAAACACTGCTGTGGCTG
GGCAATAACACCCTGGACCAAATGAGAGCTACAACCAAGATGGGCTCCG
GAGAAGGAAGAGGCTCCCTGCTGACATGCGGAGACGTGGAAGAGAACCC
AGGCCCTATGGGCGTGATCAAGCCCGACATGAAGATCAAGCTGCGGATG
GAGGGCGCCGTGAACGGCCACAAATTCGTGATCGAGGGCGACGGGAAAG
GCAAGCCCTTTGAGGGTAAGCAGACTATGGACCTGACCGTGATCGAGGG
CGCCCCCCTGCCCTTCGCTTATGACATTCTCACCACCGTGTTCGACTAC
GGTAACCGTGTCTTCGCCAAGTACCCCAAGGACATCCCTGACTACTTCA
AGCAGACCTTCCCCGAGGGCTACTCGTGGGAGCGAAGCATGACATACGA
GGACCAGGGAATCTGTATCGCTACAAACGACATCACCATGATGAAGGGT
GTGGACGACTGCTTCGTGTACAAAATCCGCTTCGACGGGGTCAACTTCC
CTGCTAATGGCCCGGTGATGCAGCGCAAGACCCTAAAGTGGGAGCCCAG
TACCGAGAAGATGTACGTGCGGGACGGCGTACTGAAGGGCGATGTTAAT
ATGGCACTGCTCTTGGAGGGAGGCGGCCACTACCGCTGCGACTTCAAGA
CCACCTACAAAGCCAAGAAGGTGGTGCAGCTTCCCGACTACCACTTCGT
GGACCACCGCATCGAGATCGTGAGCCACGACAAGGACTACAACAAAGTC
AAGCTGTACGAGCACGCCGAAGCCCACAGCGGACTACCCCGCCAGGCCG
GCTAA.

Translation of the NDV-F gene that is fused to a 2A peptide sequence from a virus Thosea asigna (T2A), followed by the coding sequence for green fluorescent protein (GFP). When translated the T2A will direct a ribosomal skip that will result in 2 independent polypeptide sequences produced, the NDV-F protein and a GFP protein.

The translation produces an amino acid sequence having at least 90% sequence identity to:

(SEQ ID NO: 5)
MGSRPSTKNPAPMMLTIRVALVLSCICPANSIDGRPLAAAGIVVTGDKA
VNIYTSSQTGSIIVKLLPNLPKDKEACAKAPLDAYNRTLTTLLTPLGDS
IRRIQESVTTSGGGRQGRL.

The SV40 poly (A) signal has a nucleic acid sequence having at least 90% identity to:

(SEQ ID NO: 6)
AACTTGTTTATTGCAGCTTATAATGGTTACAAATAAAGCAATAGCATCA
CAAATTTCACAAATAAAGCATTTTTTTCACTGCATTCTAGTTGTGGTTT
GTCCAAACTCATCAATGTATCTTA.

The sequences below represent Cas9 PAM sites located between the UL44 and UL21 CDS's of the ILT viral genome. These were used individually to evaluate insertion of the NDV-F antigen expression cassette.

Guide sequence #1:
(SEQ ID NO: 7)
GTCTGTCCAATTTATTTCATCGG.
Guide sequence #2:
(SEQ ID NO: 8)
AGGTTTCCGATGAAATAAATTGG.
Guide sequence #3:
(SEQ ID NO: 9)
TTTCATCGGAAACCTTGCACTGG.
Guide sequence #4:
(SEQ ID NO: 10)
TGAGACTACCTGCCAGTGCAAGG.

Provided herein are ssODN sequences used for directed insertion of the NDV-F antigen expression cassette. These were used to repair the cut site and destroy the PAM to prevent recutting.

The UL44-21 5′ FWD ssODN has a nucleic acid sequence with at least 90% sequence identity to:

(SEQ ID NO: 11)
GCTTTTGTGGGTGGGTTACTAGTTGCTTGCTTGGGCGCATGGTGCTGGA
AGACAACATAAACGCTCATTTAATAAATGACATTACAAACGTGCACTAC
CACCGGAGTTATTAATAGTAATCAATTACGGGGTCATTAGTTCATAGCC
CATATATGGAGTTCCGCGTTACATAACTTACGGTAAATGGCCCGCCTGG.

The UL44-21 3′ FWD ssODN has a nucleic acid sequence with at least 90% sequence identity to:

(SEQ ID NO: 12)
AAATAAAGCATTTTTTTCACTGCATTCTAGTTGTGGTTTGTCCAAACTCA
TCAATGTATCTTAGAGATCGAGTGCCGCATTGTCTGTCCAATTTATTTCA
TCGGAAACCTTGCACTGGCAGGTAGTCTCAAGCAAGCCTAAACGATATTA
TAGATTTCAATATTTCTACCAGATCCGTCCGATATTCCCAAATGAT.

### E. Codon Deoptimization

Methods of reducing a pathogen's replicative fitness are known and contemplated to be within scope of the instant invention. In some examples, the method includes deoptimizing at least one codon in a coding sequence of the pathogen, thereby generating a deoptimized coding sequence. Such deoptimization reduces replicative fitness of the pathogen. In some examples, more than one coding sequence of the pathogen is deoptimized, such as at least one, at least two, or at least 5 coding sequences, such as deoptimizing 1, 2, 3, 4, 5, 6, 7, 8, 9 or 10 coding sequences of the pathogen.

More than one codon in the one or more coding sequences can be deoptimized, such as at least 15 codons, at least 20 codons, at least 30 codons, at least 40 codons, at least 50 codons, at least 60 codons, at least 70 codons, at least 100 codons, at least 200 codons, at least 500 codons, or even at least 1000 codons, in each coding sequence. In some examples, at least 20% of the coding sequence of each desired gene is deoptimized, such as at least 30%, at least 40%, at least 50%, at least 60%, at least 70%, at least 80%, at least 90%, at least 95%, or even at least 97% deoptimized.

In particular examples, deoptimizing the codon composition alters the G+C content of a coding sequence, such as increases or decreases the G+C content by at least 10%, for example increases the G+C content of a coding sequence by at least 10%, such as at least 20%, at least 30%, at least 40%, at least 50%, at least 60%, at least 70%, at least 80%, or even by at least 90%, or decreases the G+C content of a coding sequence by at least 10%, such as at least 20%, at least 30%, at least 40%, at least 50%, at least 60%, at least 70%, at least 80%, or even by at least 90%. However, the G+C content can be altered in combination with deoptimizing one or more codons in a pathogen sequence. For example, some of the nucleotide substitutions can be made to deoptimize codons (which may or may not alter the G+C content of the sequence), and other nucleotide substitutions can be made to alter the G+C content of the sequence (which may or may result in a deoptimized codon). Altering the G+C content of the sequence may also result in a deoptimized codon, but is not required in all instances.

The replicative fitness of the pathogen can be reduced by any amount sufficient to attenuate the pathogen. In some examples, the replicative fitness of the deoptimized pathogen is reduced by at least 20%, such as at least 30%, at least 40%, at least 48%, at least 50%, at least 75%, at least 80%, at least 90%, at least 95%, or even at least 97%, as compared to replicative fitness of a pathogen (of the same species and strain) having a coding sequence with an optimizedcodon composition. Any pathogen can be attenuated using the disclosed methods. Particular examples include, but are not limited to, viruses (such as positive-strand RNA viruses, negative-strand RNA viruses, DNA viruses, and retroviruses), bacteria, fungi, and protozoa

### 1. Sequences for Deoptimization

A. RFP ORF without Stop Codon

Provided herein are methods of using a proprietary CRISPR technology for targeted replacement of the wild type (WT) ORF with a fluorescent reporter (RFP) linked to various VICOPA deoptimized ORFs. In one embodiment, the RFP ORF without stop codon has a nucleic acid sequence with at least 90% sequence identity to:

(SEQ ID NO: 13)
ATGGTGTCTAAGGGCGAAGAGCTGATTAAGGAGAACATGCACATGAAGCT
GTACATGGAGGGCACCGTGAACAACCACCACTTCAAGTGCACATCCGAGG
GCGAAGGCAAGCCCTACGAGGGCACCCAGACCATGAGAATCAAGGTGGTC
GAGGGCGGCCCTCTCCCCTTCGCCTTCGACATCCTGGCTACCAGCTTTAT
GTACGGCAGCAGAACCTTCATCAACCACACCCAGGGCATCCCCGACTTCT
TTAAGCAGTCCTTCCCTGAGGGCTTCACATGGGAGAGAGTCACCACATAC
GAAGACGGGGGCGTGCTGACCGCTACCCAGGACACCAGCCTCCAGGACGG
CTGCCTCATCTACAACGTCAAGATCAGAGGGGTGAACTTCCCATCCAACG
GCCCTGTGATGCAGAAGAAAACACTCGGCTGGGAGGCCAACACCGAGATG
CTGTACCCCGCTGACGGCGGCCTGGAAGGCAGAAGCGACATGGCCCTGAA
GCTCGTGGGCGGGGGCCACCTGATCTGCAACTTCAAGACCACATACAGAT
CCAAGAAACCCGCTAAGAACCTCAAGATGCCCGGCGTCTACTATGTGGAC
CACAGACTGGAAAGAATCAAGGAGGCCGACAAAGAGACCTACGTCGAGCA
GCACGAGGTGGCTGTGGCCAGATACTGCGACCTCCCTAGCAAACTGGGGC
ACAAACTTAAT.

### B. T2A Ribosomal Skip Coding Sequence

As discussed above, when translated the T2A will direct a ribosomal skip that will result in 2 independent polypeptide sequences produced, the NDV-F protein and a GFP protein. The T2A ribosomal skip coding sequence has a nucleic acid sequence with at least 90% sequence identity to:

(SEQ ID NO: 14)
AGAGCAGAAGGAAGGGGTTCTTTGTTGACTTGTGGAGACGTTGAGGAGAA
TCCAGGACCA

### C. Wildtype US5 Coding Sequence

Provided herein are methods for isolating RFP positive recombinant ILT with 12.5%, 25%, 50%, 75% and 95% deoptimized ORFs at the US5 locus. The wildtype US5 coding sequence has a nucleic acid sequence with at least 90% sequence identity to:

(SEQ ID NO: 15)
ATGGGGACAATGTTAGTGTTGCGCCTTTTTCTACTTGCAGTAGCGGACGCGGC
GTTGCCGACCGGCAGATTCTGCCGAGTTTGGAAGGTGCCTCCGGGAGGAACC
ATCCAAGAGAACCTGGCGGTGCTCGCGGAATCGCCGGTCACGGGACACGCGA
CATATCCGCCGCCTGAAGGCGCCGTCAGCTTTCAGATTTTTGCGGACACCCCT
ACTTTGCGCATTCGCTACGGCGCTACGGAGGACGAACTTGCACTGGAGCGCG
GGACGTCCGCCTCAGACGCGGACAACGTGACATTTTCGCTGTCATATCGCCCG
CGCCCAGAAATTCACGGAGCATACTTCACCATAGGGGTATTCGCTACTGGCC
AGAGCACGGAAAGCAGCTATTCGGTCATCAGTCGGGTCTTAGTTAACGCCTC
TCTGGAACGGTCCGTGCGCCTGGAAACGCCGTGCGATGAAAATTTTTTGCAG
AACGAGCCTACATGGGGCTCGAAGCGTTGGTTAGGCCCCCCGTCGCCTTATGT
GCGAGATAACGATGTCGCCGTGTTGACAAAAGCGCAGTACATTGGGGAGTGC
TACTCCAACTCGGCGGCCCAGACGGGGCTCACGTCTCTCAACATGACCTTTTT
CTATTCGCCTAAAAGAATAGTAAACGTCACGTGGACAACCGGCGGCCCCTCC
CCCTCGCGCATAACGGTATACTCGTCGCGGGAGAACGGGCAGCCCGTGTTGA
GGAACGTTTCTGACGGGTTCTTGGTTAAGTACACTCCCGACATTGACGGCCGG
GCCATGATAAACGTTATTGCCAATTATTCGCCGGCGGACTCCGGCAGCGTCCT
CGCGTTTACGGCCTTTAGGGAAGGAAAACTCCCATCCGCGATTCAACTGCAC
CGGATAGATATGTCCGGGACTGAGCCGCCGGGGACTGAAACGACCTTCGACT
GTCAAAAAATGATAGAAACCCCGTACCGAGCGCTCGGGAGCAATGTTCCCAG
GGACGACTCTATCCGTCCGGGGGCCACTCTGCCTCCGTTCGATACCGCAGCAC
CTGATTTCGATACAGGTACTTCCCCGACCCCCACTACCGTGCCAGAGCCAGCC
ATTACTACACTCATACCGCGCAGCACTAGCGATATGGGATTCTTCTCCACGGC
ACGTGCTACCGGATCAGAAACTCTTTCGGTACCCGTCCAGGAAACGGATAGA
ACTCTTTCGACAACTCCTCTTACCCTTCCACTGACTCCCGGTGAGTCAGAAAA
TACACTGTTTCCTACGACCGCGCCGGGGATTTCTACCGAGACCCCGAGCGCG
GCACATGAAACTACACAGACCCAGAGTGCAGAAACGGTGGTCTTTACTCAGA
GTCCGAGTACCGAGTCGGAAACCGCGCGGTCCCAGAGTCAGGAACCGTGGTA
TTTTACTCAGACTCCGAGTACTGAACAGGCGGCTCTTACTCAGACGCAGATCG
CAGAAACGGAGGCGTTGTTTACTCAGACTCCGAGTGCTGAACAGATGACTTT
TACTCAGACTCCGGGTGCAGAAACCGAGGCACCTGCCCAGACCCCGAGCACG
ATACCCGAGATATTTACTCAGTCTCGTAGCACGCCCCCCGAAACCGCTCGCGC
TCCGAGCGCGGCGCCGGAGGTTTTTACACAGAGTTCGAGTACGGTAACGGAG
GTGTTTACTCAGACCCCGAGCACGGTACCGAAAACTACTCTGAGTTCGAGTA
CTGAACCGGCGATTTTTACTCGGACTCAGAGCGCGGGAACTGAGGCCTTTACT
CAGACTTCGAGTGCCGAGCCGGACACTATGCGAACTCAGAGTACTGAAACAC
ACTTTTTCACTCAGGCCCCGAGTACGGTACCGAAAGCTACTCAGACTCCGAGT
ACAGAGCCGGAGGTGTTGACTCAGAGTCCGAGTACCGAACCTGTGCCTTTCA
CCCGGACTCTGGGCGCAGAGCCGGAAATTACTCAGACCCCGAGCGCGGCACC
GGAGGTTTATACTCGGAGTTCGAGTACGATGCCAGAAACTGCACAGAGCACA
CCCCTGGCCTCGCAAAACCCTACCAGTTCGGGAACCGGGACGCATAATACTG
AACCGAGGACTTATCCAGTGCAAACGACACCACATACCCAGAAACTCTACAC
AGAAAATAAGACTTTATCGTTTCCTACTGTTGTTTCAGAATTCCATGAGATGT
CGACGGCAGAGTCGCAGACGCCCCTATTGGACGTCAAAATTGTAGAGGTGAA
GTTTTCAAACGATGGCGAAGTAACGGCGACTTGCGTTTCCACCGTCAAATCTC
CCTATAGGGTAGAAACTAATTGGAAAGTAGACCTCGTAGATGTAATGGATGA
AATTTCTGGGAACAGTCCCGCCGGGGTTTTTAACAGTAATGAGAAATGGCAG
AAACAGCTGTACTACAGAGTAACCGATGGAAGAACATCGGTCCAGCTAATGT
GCCTGTCGTGCACGAGCCATTCTCCGGAACCTTACTGTCTTTTCGACACGTCT
CTTATAGCGAGGGAAAAAGATATCGCGCCAGAGTTATACTTTACCTCTGATCC
GCAAACGGCATACTGCACAATAACTCTGCCGTCCGGCGTTGTTCCGAGATTCG
AATGGAGCCTTAATAATGTTTCACTGCCGGAATATTTGACGGCCACGACCGTT
GTTTCGCATACCGCTGGCCAAAGTACAGTGTGGAAGAGCAGCGCGAGAGCAG
GCGAGGCGTGGATTTCTGGCCGGGGAGGCAATATATACGAATGCACCGTCCT
CATCTCAGACGGCACTCGCGTTACTACGCGAAAGGAGAGGTGCTTAACAAAC
ACATGGATTGCGGTGGAAAACGGTGCTGCTCAGGCGCAGCTGTATTCACTCTT
TTCTGGACTTGTGTCAGGATTATGCGGGAGCATATCTGCTTTGTACGCAACGC
TATGGACCGCCATTTATTTTTGA.

### D. Cas9 PAM Sequences

CRISPR/Cas9 editing technologies were used to generate rLT/F. Briefly, LMH cells were transfected with Ribonucleoprotein Complexes (RNPs) Cas9/Cpf1 with complexed guide as well as repair templates containing a promoter, NDV fusion gene, and selection cassette. The guides were directed to cut at PAM sites in the targeted insertion sites. The sites targeted for insertion include UL45/46, sORF-1, UL44/21 and UL3/4. Guides were selected based on guide cutting efficiency assays to detect the presence of non-homologous end joining (NHEJ).

The US5 3′ Cas9 PAM sequence is a nucleic acid sequence having at least 90% sequence identity to:

(SEQ ID NO: 16)
GCGTTACTACGCGAAAGGAGAGG.

The US5 5′ Cas9 PAM sequence is a nucleic acid sequence having at least 90% sequence identity to:

(SEQ ID NO: 17)
CCGCGTCCGCTACTGCAAGTAGG.

### E. Sequences for Inserting the Deoptimized Sequences

Provided herein are methods of inserting the deoptimized sequences into the rLT vector. The US5 3′ homology arm directs insertion of the deoptimized US5 ORF sequence into the rLT vector. The US5 3′ homology arm has a nucleic acid sequence with at least 90% identity to:

(SEQ ID NO: 18)
AGCGCGAGAGCAGGCGAGGCGTGGATTTCTGGCCGGGGAGGCAATATATA
CGAATGCACCGTCCTCATCTCAGACGGCACTCGCGTTACTACGCGCAAGG
AGAGATGCTTAACAAACACATGGATTGCGGTGGAAAACGGTGCTGCTCAG
GCGCAGCTGTATTCACTCTTTTCTGGACTTGTGTCAGGATTATGCGGG.

The RFP-T2A/US5 5′ homology arm was used to anneal the RFP cassette to the US5 deoptimized cassette. This homology arm also destroys the US5 5′ PAM sequence. The RFP-T2A/US5 5′ homology arm has a nucleic acid sequence with at least 90% identity to:

(SEQ ID NO: 19)
CTCCCTAGCAAACTGGGGCACAAACTTAATAGAGCAGAAGGAAGGGGTTC
TTTGTTGACTTGTGGAGACGTTGAGGAGAATCCAGGACCAATGGGGACAA
TGTTAGTGTTGCGCCTTTTTCTACTTGCAGTAGCGGACGCGGCGTTGCCG
ACCGGCAGATTCTGCCGAGTTTGGAAGGTGCCTCCGGGAGGAACCATCC
A.

The RFP 5′ homology arm was used to direct integration of the RFP cassette directly in front of the US5 start codon. The RFP 5′ homology arm has a nucleic acid sequence with at least 90% identity to:

(SEQ ID NO: 20)
TTTGCGTGTCTGTGCTAAACCATGGCGTGTGCGGGTGAAACCGTAAATTA
CGTGATAATAAATAGCATAGGAGTTGGCGTGCAGCGTATTTCGCCGAGAG
AAGGGGTTCTTTGTTGACTTGTGGAGACGTTGTGGATGGTGTCTAAGGGC
GAAGAGCTGATTAAGGAGAACATGCACATGAAGCTGTACATGGAGGGCA
C.

The Cas9 PAM site that is part of the T2A sequence has a nucleic acid sequence with at least 90% identity to:

(SEQ ID NO: 21)
AGGGGTTCTTTGTTGACTTGTGG.

This sequence is flanking the RFP ORF and will be used to remove the RFP expression cassette prior to commercial use of the vaccine vector.

2. Deoptimized US5 Coding Sequences Inserted into the ILT Genome

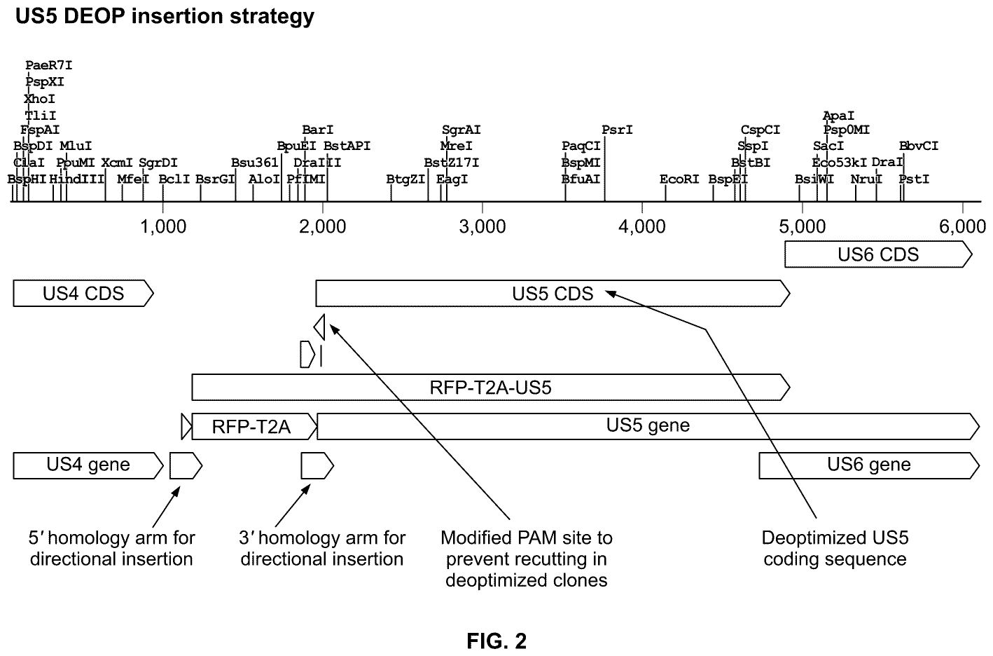

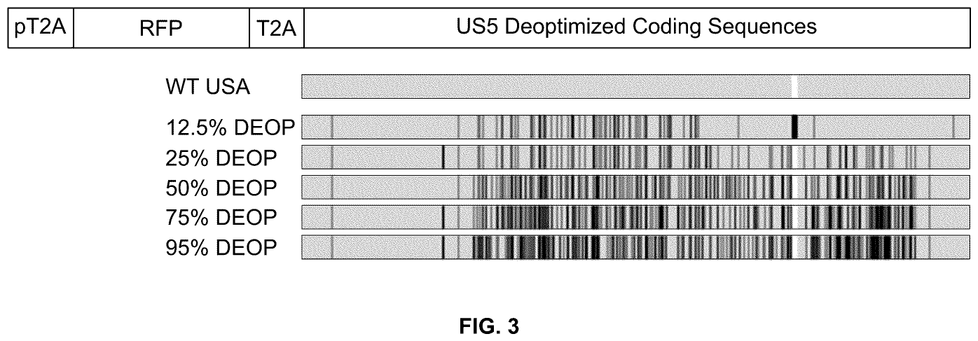

The sequences below represent deoptimized US5 coding sequences that were successfully inserted into the ILT genome replacing the wild type US5 coding sequence (CDS), as illustrated in FIG. 2. The sequences were deoptimized as illustrated in FIG. 3.

### A. 12.5% US5 Deoptimized Coding Sequence

Provided herein is the 12.5% US5 Deoptimized coding sequence having at least 90% sequence identity to:

(SEQ ID NO: 22)
ATGGGGACAATGTTAGTGTTGCGCCTTTTTCTACTTGCAGTAGCGGACGCGGC
GTTGCCGACCGGCAGATTCTGCCGAGTTTGGAAGGTGCCTCCGGGAGGAACC
ATCCAAGAGAACCTGGCAGTGCTCGCGGAATCGCCGGTCACGGGACACGCGA
CATATCCGCCGCCTGAAGGCGCCGTCAGCTTTCAGATTTTTGCGGACACCCCT
ACTTTGCGCATTCGCTACGGTGCTACGGAAGACGAACTTGCACTGGAACGCG
GGACGTCCGCCTCAGACGCGGACAACGTGACATTTTCGCTGTCATATCGCCCG
CGCCCAGAAATTCACGGTGCATACTTCACCATAGGGGTATTCGCGACTGGTC
AGAGCACGGAAAGCAGCTATTCGGTCATCAGTCGGGTCTTAGTAAACGCGTC
TCTGGAACGGTCCGTACGCCTGGAAACGCCTTGCGATGAAAATTTTTTGCAGA
ACGAGCCTACATGGGGCTCGAAGCGTTGGTTAGGCCCCCCGTCGCCTTACGT
ACGAGATAACGACGTCGCCGTGTTGACAAAAGCGCAGTACATAGGCGAGTGT
TACTCCAACTCGGCAGCCCAAACGGGTCTCACGTCTCTCAACATGACCTTTTT
CTATAGTCCTAAAAGAATAGTAAACGTCACGTGGACGACCGGCGGCCCCTCC
CCCTCGCGTATAACGGTATACTCGTCGCGGGAGAACGGGCAGCCGGTGTTGA
GGAACGTTTCGGACGGATTCCTAGTTAAGTACACTCCCGACATTGACGGCCG
GGCGATGATAAACGTTATTGCCAATTATTCGCCAGCGGACTCCGGCAGCGTC
CTCGCGTTTACGGCCTTTAGGGAAGGTAAATTACCATCCGCGATACAACTGCA
CCGGATCGATATGTCCGGTACTGAGCCGCCGGGGACGGAAACGACGTTCGAC
TGTCAAAAAATGATCGAAACCCCGTACCGAGCGCTAGGGTCGAATGTACCCA
GGGACGATTCGATCCGTCCGGGGGCCACTCTGCCGCCGTTCGATACCGCAGC
ACCTGATTTCGATACGGGTACGTCCCCGACCCCCACTACCGTGCCAGAGCCG
GCCATTACTACCCTCATACCGCGCTCGACTAGCGATATGGGATTCTTTTCCAC
GGCTAGGGCTACCGGATCAGAAACTCTTTCGGTACCCGTCCAGGAAACGGAT
AGAACTCTTTCGACAACTCCTCTTACGCTTCCGCTGACTCCCGGTGAGTCGGA
AAATACACTGTTTCCGACGACCGCACCGGGTATTTCGACCGAGACCCCGAGC
GCGGCACATGAAACTACACAGACCCAGAGTGCAGAAACGGTGGTCTTTACTC
AGTCGCCGAGTACCGAGTCGGAAACCGCGCGGTCCCAGTCGCAAGAACCGTG
GTATTTTACTCAAACTCCTAGTACTGAACAAGCGGCTCTTACTCAGACGCAGA
TCGCAGAAACGGAGGCGTTGTTTACTCAGACTCCGAGTGCTGAACAGATGAC
TTTTACTCAGACTCCGGGTGCAGAAACCGAGGCACCTGCCCAGACCCCGAGC
ACGATACCCGAGATATTTACTCAGTCTCGTAGCACGCCCCCCGAAACCGCTCG
CGCTCCGAGCGCGGCGCCGGAGGTTTTTACACAGAGTTCGTCGACGGTAACG
GAGGTGTTTACTCAGACCCCGAGCACGGTACCGAAAACTACTCTGAGTTCGA
GTACTGAACCGGCGATTTTTACTCGGACTCAGAGCGCGGGAACTGAGGCCTT
TACTCAGACTTCGAGTGCCGAGCCGGACACTATGCGAACTCAGAGTACTGAA
ACACACTTTTTCACTCAGGCCCCGAGTACGGTACCGAAAGCTACTCAGACTCC
GAGTACAGAGCCGGAGGTGTTGACTCAGAGTCCGAGTACCGAACCTGTGCCT
TTCACCCGGACTCTGGGCGCAGAGCCGGAAATTACTCAGACTCCGAGTACGG
TACCGGAAATTACTCAGACCCCGAGCGCGGCACCGGAGGTTTATACTCGGAG
TTCGAGTACGATGCCAGAAACTGCACAGAGCACACCCCTGACCTCGCAAAAC
CCTACCAGTTCGGGAACCGGGACGCATAATACTGAACCGAGGACTTATCCAG
TGCAAACGACACCACATACCCAGAAACTCTACACAGAAAATAAGACTTTATC
GTTTCCTACTGTTGTTTCAGAATTCCATGAGATGTCGACGGCAGAGTCGCAGA
CGCCCCTATTGGACGTCAAAATTGTAGAGGTGAAGTTTTCAAACGATGGCGA
AGTAACGGCGACTTGCGTTTCCACCGTCAAATCTCCCTATAGGGTAGAAACTA
ATTGGAAAGTAGACCTCGTAGATGTAATGGATGAAATTTCTGGGAACAGTCC
CGCCGGGGTTTTTAACAGTAATGAGAAATGGCAGAAACAGCTGTACTACAGA
GTAACCGATGGAAGAACATCGGTCCAGCTAATGTGCCTGTCGTGCACGAGCC
ATTCTCCGGAACCTTACTGTCTTTTCGACACGTCTCTTATAGCGAGGGAAAAA
GATATCGCGCCAGAGTTATACTTTACCTCTGATCCGCAAACGGCATACTGCAC
AATAACTCTGCCGTCCGGCGTTGTTCCGAGATTCGAATGGAGCCTTAATAATG
TTTCACTGCCGGAATATTTGACGGCCACGACCGTTGTTTCGCATACCGCTGGC
CAAAGTACAGTGTGGAAGAGCAGCGCGAGAGCAGGCGAGGCGTGGATTTCT
GGCCGGGGAGGCAATATATACGAATGCACCGTCCTCATCTCAGACGGCACTC
GCGTTACTACGCGAAAGGAGAGATGCTTAACAAACACATGGATTGCGGTGGA
AAACGGTGCTGCTCAGGCGCAGCTGTATTCACTCTTTTCTGGACTTGTGTCAG
GATTATGCGGGAGCATATCTGCTTTGTACGCAACGCTATGGACCGCCATTTAT
TTTTGA.

### b. 25% US5 Deoptimized Coding Sequence

Provided herein is the 25% US5 Deoptimized coding sequence having at least 90% sequence identity to:

(SEQ ID NO: 23)
ATGGGGACAATGTTAGTGTTGCGCCTTTTTCTACTAGCTGTTGCGGACGCGGC
GTTGCCGACCGGCAGATTCTGCCGAGTTTGGAAGGTGCCTCCGGGAGGAACC
ATCCAAGAGAACCTGGCAGTGCTCGCGGAATCGCCGGTCACGGGACACGCGA
CATATCCGCCGCCTGAAGGCGCCGTCAGCTTTCAGATTTTTGCGGACACCCCT
ACTTTGCGCATTCGCTACGGTGCTACGGAAGACGAACTTGCACTGGAACGCG
GGACGTCCGCCTCAGACGCGGACAACGTGACATTTTCGCTGTCATATCGCCCG
CGCCCAGAAATTCACGGTGCATACTTCACCATAGGGGTATTCGCGACTGGTC
AGAGCACGGAAAGCAGCTATTCGGTCATCAGTCGGGTCTTAGTAAACGCGTC
TCTGGAACGGTCCGTACGCCTGGAAACGCCTTGCGATGAAAATTTTTTGCAGA
ACGAGCCTACATGGGGCTCGAAGCGTTGGTTAGGCCCCCCGTCGCCTTACGT
ACGAGATAACGACGTCGCCGTGTTGACAAAAGCGCAGTACATAGGCGAGTGT
TACTCCAACTCGGCAGCCCAAACGGGTCTCACGTCTCTCAACATGACCTTTTT
CTATAGTCCTAAAAGAATAGTAAACGTCACGTGGACGACCGGCGGCCCCTCC
CCCTCGCGTATAACGGTATACTCGTCGCGGGAGAACGGGCAGCCGGTGTTGA
GGAACGTTTCGGACGGGTTCCTAGTTAAGTACACTCCCGACATTGACGGCCG
GGCGATGATAAACGTTATTGCCAATTATTCGCCAGCGGACTCCGGCAGCGTC
CTCGCGTTTACGGCCTTTAGGGAAGGTAAATTACCATCCGCGATACAACTGCA
CCGGATCGATATGTCCGGTACTGAGCCGCCGGGGACGGAAACGACGTTCGAC
TGTCAAAAAATGATCGAAACCCCGTACCGAGCGCTAGGGTCGAATGTACCCA
GGGACGATTCGATCCGTCCGGGGGCCACTCTGCCGCCGTTCGATACCGCAGC
ACCTGATTTCGATACGGGTACGTCCCCGACCCCCACTACCGTGCCAGAGCCG
GCCATTACTACCCTCATACCGCGCTCGACTAGCGATATGGGATTCTTTTCCAC
GGCTAGGGCTACCGGATCAGAAACTCTTTCGGTACCCGTCCAGGAAACGGAT
AGAACTCTTTCGACAACTCCTCTTACGCTTCCGCTGACTCCCGGTGAGTCGGA
AAATACACTGTTTCCGACGACCGCACCGGGTATTTCGACCGAGACCCCGAGC
GCGGCACATGAAACTACACAGACCCAGAGTGCAGAAACGGTGGTCTTTACTC
AGTCGCCGAGTACCGAGTCGGAAACCGCGCGATCCCAGTCGCAAGAACCGTG
GTATTTTACTCAAACTCCTAGTACTGAACAAGCGGCACTTACTCAGACGCAGA
TCGCAGAAACGGAGGCGCTATTTACGCAGACTCCGAGTGCGGAACAAATGAC
TTTTACTCAGACTCCGGGTGCAGAAACCGAGGCGCCTGCCCAGACCCCGAGC
ACGATACCCGAGATATTTACTCAGTCTCGTAGCACGCCCCCCGAAACCGCTCG
CGCTCCTAGCGCGGCGCCGGAGGTTTTTACACAGAGTTCGTCGACGGTAACG
GAGGTGTTTACTCAGACCCCGAGCACGGTACCGAAAACTACTCTATCGTCGA
GTACTGAACCGGCGATTTTTACTCGGACTCAGAGCGCGGGAACTGAGGCCTT
TACTCAGACTTCGAGTGCCGAGCCGGACACTATGCGAACTCAGAGTACTGAA
ACACATTTTTTCACTCAAGCCCCGAGTACGGTACCGAAAGCTACTCAGACTCC
GAGTACAGAGCCGGAGGTGCTAACTCAGAGTCCGAGTACCGAACCTGTGCCG
TTCACCCGGACGCTGGGCGCCGAGCCGGAAATTACTCAGACCCCGAGCGCGG
CACCGGAAGTTTATACTCGGAGTTCGAGTACGATGCCAGAAACTGCACAGAG
CACACCCCTGGCGTCGCAAAACCCTACCAGTTCGGGAACCGGGACGCATAAT
ACTGAACCGAGGACTTATCCAGTGCAAACGACTCCGCATACCCAGAAGCTCT
ACACAGAAAATAAGACTTTATCGTTTCCTACTGTTGTTTCAGAATTTCATGAA
ATGTCGACGGCAGAGTCGCAGACGCCGCTACTCGACGTCAAAATTGTAGAGG
TGAAGTTTTCAAACGATGGTGAGGTAACGGCGACATGCGTTTCCACCGTCAA
ATCTCCCTATAGGGTAGAAACTAATTGGAAAGTAGACCTAGTAGATGTAATG
GACGAAATTTCTGGGAATAGTCCCGCGGGGGTTTTTAACAGTAATGAGAAAT
GGCAGAAACAACTGTACTACCGCGTAACCGATGGAAGAACATCGGTCCAGCT
AATGTGCCTATCGTGCACGAGCCATAGTCCGGAACCTTACTGTCTTTTCGACA
CGTCTCTTATAGCGAGGGAAAAAGATATCGCGCCAGAGTTATACTTTACGTCT
GATCCGCAAACGGCATACTGTACAATAACTCTGCCGTCCGGCGTCGTTCCGA
GATTCGAATGGAGCCTTAATAATGTTTCACTGCCGGAATATTTGACGGCCACG
ACCGTTGTTTCGCATACAGCTGGCCAAAGTACAGTGTGGAAGAGCAGCGCGA
GAGCAGGCGAGGCGTGGATTTCTGGCCGGGGAGGCAATATATACGAATGCAC
CGTCCTCATCTCAGACGGCACTCGCGTTACTACGCGAAAGGAGAGGTGCTTA
ACAAACACATGGATTGCGGTGGAAAACGGTGCTGCTCAGGCGCAGCTGTATT
CACTCTTTTCTGGACTTGTGTCAGGATTATGCGGGAGCATATCTGCTTTGTAC
GCAACGCTATGGACCGCCATTTATTTTTGA.

### c. 50% US5 Deoptimized Coding Sequence

Provided herein is the 50% US5 Deoptimized coding sequence having at least 90% sequence identity to:

(SEQ ID NO: 24)
ATGGGGACAATGTTAGTGTTGCGCCTTTTTCTACTTGCAGTAGCGGACGCGGC
GTTGCCGACCGGCAGATTCTGCCGAGTTTGGAAGGTGCCTCCGGGAGGAACC
ATCCAAGAGAACCTGGCAGTGCTCGCGGAATCGCCGGTCACGGGACACGCGA
CATATCCGCCGCCTGAAGGCGCCGTCAGCTTTCAGATTTTTGCGGACACGCCT
ACTTTGCGCATTCGCTACGGTGCTACGGAAGACGAACTTGCACTCGAACGCG
GGACGTCCGCGTCCGACGCGGACAACGTGACATTTTCGCTGTCGTATCGCCCG
CGCCCAGAAATTCACGGTGCATACTTCACCATAGGCGTATTCGCTACCGGTCA
GAGCACGGAATCGAGCTATTCGGTCATCTCGCGGGTCTTAGTAAACGCGTCA
CTGGAACGGTCCGTACGACTGGAAACGCCTTGCGATGAAAACTTTCTCCAGA
ACGAGCCTACATGGGGTTCGAAACGTTGGTTAGGCCCCCCGAGTCCTTACGT
ACGAGATAACGACGTCGCCGTGTTGACAAAAGCGCAATACATTGGCGAGTGT
TATTCGAACTCGGCAGCCCAAACGGGTCTAACGTCTCTAAACATGACCTTTTT
CTATAGTCCGAAAAGAATAGTAAACGTCACGTGGACGACCGGCGGCCCCTCC
CCGTCGCGTATAACGGTATACTCGTCGCGCGAGAACGGTCAGCCGGTGTTGA
GGAACGTTTCGGACGGATTCCTAGTTAAGTACACTCCCGACATTGACGGCAG
GGCGATGATAAACGTAATTGCCAATTATTCGCCAGCGGACTCGGGCTCGGTC
CTCGCGTTTACGGCCTTTAGGGAAGGTAAACTCCCGTCCGCGATACAATTACA
CCGTATCGATATGTCCGGGACTGAACCGCCGGGGACTGAAACGACGTTCGAT
TGTCAAAAAATGATCGAAACCCCGTACCGAGCGCTAGGGTCGAATGTACCCA
GGGACGACTCTATACGTCCGGGGGCGACTCTGCCTCCATTCGATACCGCAGC
ACCGGATTTCGATACGGGTACTTCCCCGACTCCCACTACCGTGCCAGAGCCGG
CCATTACGACCCTCATACCGCGCTCGACTTCCGATATGGGATTCTTCTCCACC
GCTCGTGCGACCGGATCAGAAACTCTTTCGGTACCCGTACAGGAAACGGATC
GTACGCTTTCGACAACTCCTCTTACGCTTCCGCTGACTCCTGGTGAGTCGGAA
AATACACTGTTTCCGACGACCGCACCGGGTATTTCTACCGAGACCCCGAGCG
CGGCACATGAAACTACACAGACCCAAAGTGCAGAAACGGTCGTCTTTACTCA
GAGTCCATCGACCGAGTCGGAAACCGCGCGATCCCAGTCGCAAGAACCGTGG
TATTTTACTCAAACTCCTAGTACTGAACAAGCGGCACTTACTCAGACGCAAAT
CGCAGAAACGGAGGCGCTATTTACGCAGACTCCGAGTGCGGAACAAATGACT
TTTACTCAGACGCCCGGTGCAGAAACCGAAGCGCCTGCCCAGACCCCGAGCA
CGATACCGGAGATATTTACTCAGTCGCGATCGACGCCCCCGGAAACGGCTCG
CGCTCCTAGCGCGGCGCCGGAAGTCTTTACACAGAGTTCGTCGACGGTAACC
GAGGTGTTTACTCAGACCCCTAGCACGGTACCGAAAACTACCCTATCGTCGA
GTACTGAACCGGCGATTTTTACGCGGACTCAAAGCGCGGGAACTGAGGCCTT
TACTCAGACTTCGAGTGCCGAACCGGATACTATGCGAACTCAGAGTACTGAA
ACACATTTTTTCACGCAAGCCCCAAGTACGGTACCGAAAGCTACTCAGACTCC
GAGTACAGAGCCGGAGGTGCTAACGCAAAGTCCGAGTACCGAACCTGTGCCG
TTCACCCGGACGCTAGGCGCCGAGCCGGAAATTACTCAGACCCCGAGCGCGG
CACCGGAGGTTTATACGCGAAGTTCGAGTACGATGCCAGAAACTGCACAATC
GACACCCCTGGCGTCGCAAAACCCTACCTCGTCGGGTACCGGGACGCATAAT
ACTGAACCGAGGACTTATCCAGTGCAAACGACTCCGCATACCCAGAAGCTCT
ACACAGAAAATAAGACTTTATCGTTTCCTACGGTTGTATCGGAATTTCATGAA
ATGTCGACGGCAGAGTCGCAGACGCCGCTACTCGACGTCAAGATTGTAGAGG
TGAAGTTTTCAAACGATGGTGAGGTAACGGCGACATGCGTTTCCACCGTCAA
GTCGCCCTATAGGGTAGAAACTAATTGGAAAGTAGACCTAGTAGATGTAATG
GACGAGATTTCTGGGAATAGTCCCGCGGGGGTTTTTAACAGTAACGAGAAGT
GGCAAAAACAACTGTACTACCGCGTAACGGATGGAAGAACATCGGTCCAGCT
AATGTGTCTATCGTGTACGAGCCATTCTCCGGAACCTTATTGTCTTTTCGACA
CGTCGCTTATCGCGAGGGAAAAAGATATAGCGCCCGAATTATACTTTACGTCT
GATCCGCAAACGGCGTACTGTACGATAACTCTACCGTCCGGCGTCGTTCCGA
GATTCGAATGGAGCCTTAATAATGTTTCACTGCCGGAATATTTGACGGCCACG
ACCGTTGTTTCGCATACAGCTGGCCAAAGTACAGTGTGGAAGAGCAGCGCGA
GAGCAGGCGAGGCGTGGATTTCTGGCCGGGGAGGCAATATATACGAATGCAC
CGTCCTCATCTCAGACGGCACTCGCGTTACTACGCGAAAGGAGAGGTGCTTA
ACAAACACATGGATTGCGGTGGAAAACGGTGCTGCTCAGGCGCAGCTGTATT
CACTCTTTTCTGGACTTGTGTCAGGATTATGCGGGAGCATATCTGCTTTGTAC
GCAACGCTATGGACCGCCATTTATTTTTGA.

### d. 75% US5 Deoptimized Coding Sequence

Provided herein is the 75% US5 Deoptimized coding sequence having at least 90% sequence identity to:

(SEQ ID NO: 25)
ATGGGGACAATGTTAGTGTTGCGCCTTTTTCTACTAGCTGTTGCGGACGCGGC
GTTGCCGACCGGCAGATTCTGCCGAGTTTGGAAGGTGCCTCCGGGAGGAACC
ATCCAAGAGAACCTGGCAGTGCTCGCGGAATCGCCGGTCACGGGACACGCGA
CATATCCGCCGCCTGAAGGCGCCGTCAGCTTTCAGATTTTTGCGGACACGCCT
ACTTTGCGCATTCGGTACGGCGCTACGGAAGACGAACTTGCACTCGAACGCG
GGACGTCCGCGTCCGACGCGGACAACGTGACATTTTCGCTATCATATCGCCCG
CGCCCAGAAATTCACGGTGCGTACTTTACCATAGGGGTATTCGCGACTGGTCA
GTCGACGGAATCGAGCTATTCGGTAATCTCGCGGGTATTAGTAAACGCGTCA
CTGGAACGGTCCGTACGACTGGAAACGCCTTGCGATGAAAACTTTCTCCAGA
ACGAACCTACATGGGGTTCGAAACGTTGGTTAGGGCCCCCGAGTCCTTACGT
ACGAGATAACGACGTAGCCGTATTGACGAAAGCGCAGTATATAGGCGAATGC
TACTCGAATTCGGCAGCCCAAACGGGTCTAACGTCTCTAAATATGACCTTTTT
CTATAGTCCGAAAAGAATAGTAAACGTCACGTGGACGACCGGCGGCCCCTCC
CCCTCGCGTATAACGGTATACTCGTCGCGGGAGAACGGTCAACCGGTGTTGA
GGAACGTTTCTGACGGATTCCTAGTTAAGTATACGCCCGACATTGACGGCAG
GGCGATGATAAACGTTATCGCCAATTATTCGCCAGCGGACTCGGGCAGCGTC
CTCGCGTTTACGGCCTTTCGCGAAGGTAAGTTACCAAGTGCGATACAATTACA
CCGGATCGATATGTCCGGTACTGAGCCGCCGGGGACTGAAACGACGTTCGAT
TGTCAAAAAATGATCGAAACCCCGTACCGAGCGCTCGGGTCGAACGTTCCGA
GGGACGACTCTATACGTCCGGGTGCCACCCTGCCGCCATTCGATACGGCAGC
ACCTGATTTCGATACAGGTACGAGTCCGACTCCGACTACCGTACCAGAGCCA
GCGATTACGACCCTCATACCGCGCTCGACTTCCGATATGGGATTCTTCTCCAC
CGCTCGTGCGACCGGATCAGAAACTCTATCGGTACCCGTACAGGAAACGGAT
AGAACGCTTTCGACAACTCCGCTTACGCTTCCGTTAACGCCTGGTGAATCAGA
AAATACACTATTTCCGACGACCGCGCCGGGGATTTCTACCGAGACCCCGAGC
GCGGCACATGAAACTACACAAACGCAAAGTGCAGAAACGGTCGTCTTTACTC
AAAGTCCGTCGACCGAGTCGGAAACCGCGCGATCCCAATCGCAAGAACCGTG
GTATTTTACTCAAACTCCTTCGACTGAACAAGCGGCACTTACTCAGACGCAAA
TAGCGGAAACGGAAGCGTTGTTTACTCAGACTCCGAGTGCGGAACAAATGAC
TTTTACTCAGACTCCGGGTGCAGAAACCGAAGCGCCTGCCCAGACCCCGAGC
ACGATACCCGAAATATTTACTCAGTCTCGTAGCACGCCCCCCGAAACGGCTC
GCGCTCCGAGCGCGGCGCCGGAAGTCTTTACACAGAGTTCGAGTACGGTAAC
CGAGGTGTTTACTCAGACCCCTAGCACGGTACCGAAAACTACCCTGTCGTCG
AGTACTGAACCGGCGATTTTTACGCGTACTCAGTCGGCCGGAACTGAAGCCTT
TACTCAGACTTCGAGTGCCGAGCCGGACACGATGCGAACTCAGAGTACTGAA
ACACATTTTTTCACGCAAGCCCCGAGTACGGTACCGAAAGCTACTCAGACTCC
GAGTACAGAGCCGGAGGTGCTAACGCAGTCGCCAAGTACCGAACCTGTGCCG
TTTACCCGGACTCTAGGCGCCGAGCCGGAAATTACTCAGACCCCGAGCGCGG
CACCGGAGGTTTATACGCGAAGTTCGAGTACGATGCCAGAAACTGCACAAAG
CACTCCGTTAGCGTCGCAAAACCCGACTTCGTCGGGTACCGGGACGCATAAT
ACTGAACCGAGGACTTATCCGGTGCAAACGACTCCACATACGCAAAAGCTAT
ACACGGAAAATAAGACGTTATCGTTTCCTACTGTCGTTTCGGAATTTCACGAA
ATGTCGACCGCAGAGTCGCAGACGCCCCTATTGGACGTAAAGATTGTAGAGG
TGAAGTTTTCGAACGATGGTGAGGTAACGGCGACATGCGTTTCCACCGTTAA
GTCGCCCTATAGGGTAGAAACTAATTGGAAAGTAGACCTAGTAGATGTAATG
GACGAGATTTCTGGTAATTCGCCCGCGGGGGTTTTCAATAGTAACGAAAAGT
GGCAAAAACAACTATATTACCGCGTAACGGATGGTAGAACGTCGGTCCAATT
GATGTGTCTATCGTGTACGAGCCATTCTCCGGAACCGTACTGTCTATTCGACA
CGTCGCTAATAGCGAGGGAAAAAGACATAGCGCCCGAGTTATACTTTACGTC
TGATCCGCAAACCGCATATTGTACGATTACTCTACCGTCCGGCGTCGTTCCGA
GATTCGAATGGAGCCTTAATAATGTTTCACTGCCGGAATATTTGACGGCCACG
ACCGTTGTTTCGCATACAGCTGGCCAAAGTACAGTGTGGAAGAGCAGCGCGA
GAGCAGGCGAGGCGTGGATTTCTGGCCGGGGAGGCAATATATACGAATGCAC
CGTCCTCATCTCAGACGGCACTCGCGTTACTACGCGAAAGGAGAGGTGCTTA
ACAAACACATGGATTGCGGTGGAAAACGGTGCTGCTCAGGCGCAGCTGTATT
CACTCTTTTCTGGACTTGTGTCAGGATTATGCGGGAGCATATCTGCTTTGTAC
GCAACGCTATGGACCGCCATTTATTTTTGA.

### d. 95% US5 Deoptimized Coding Sequence

Provided herein is the 95% US5 Deoptimized coding sequence having at least 90% sequence identity to:

(SEQ ID NO: 26)
ATGGGGACAATGTTAGTGTTGCGCCTTTTTCTACTAGCTGTTGCGGACGCGGC
GTTGCCGACCGGCAGATTCTGCCGAGTTTGGAAGGTGCCTCCGGGAGGAACC
ATCCAAGAGAACCTGGCAGTGCTCGCGGAATCGCCGGTCACGGGACACGCGA
CATATCCGCCGCCTGAAGGCGCCGTCAGCTTTCAGATTTTTGCGGATACGCCT
ACGTTGCGCATTCGGTACGGCGCGACCGAAGACGAACTAGCGCTGGAACGCG
GGACGTCCGCGTCCGACGCCGACAACGTAACGTTTTCGCTATCATATCGCCCG
CGCCCAGAAATTCACGGTGCATACTTTACCATAGGGGTATTCGCTACTGGCCA
GAGCACGGAAAGCTCGTATTCGGTAATCTCGCGGGTATTAGTAAACGCGTCA
CTCGAACGGTCCGTGCGCCTGGAAACGCCTTGCGATGAAAACTTTCTCCAAA
ACGAACCTACTTGGGGTTCGAAGCGGTGGTTAGGGCCCCCGAGTCCTTACGT
GCGCGATAACGACGTAGCCGTGTTGACAAAAGCGCAGTATATAGGCGAATGC
TACTCGAATTCGGCAGCGCAAACGGGTCTAACGTCTCTAAATATGACGTTTTT
CTATAGTCCTAAACGTATCGTAAACGTCACGTGGACAACCGGCGGCCCCAGT
CCGTCGCGCATAACGGTATATTCGTCGCGGGAGAACGGTCAGCCGGTATTGA
GGAACGTTTCTGACGGATTCCTAGTTAAGTATACGCCCGATATTGACGGTAGG
GCGATGATAAACGTTATCGCGAATTATTCGCCAGCGGATTCGGGTTCGGTCCT
CGCGTTTACGGCCTTTCGCGAAGGTAAGTTACCAAGTGCGATACAATTACACC
GTATCGATATGTCCGGGACTGAGCCGCCGGGGACTGAAACGACCTTCGATTG
TCAAAAAATGATCGAAACCCCGTATAGGGCGCTAGGGAGCAATGTTCCGCGA
GACGACTCTATACGTCCGGGTGCCACTCTGCCGCCATTCGATACGGCAGCACC
GGATTTCGATACAGGTACTTCCCCGACTCCCACTACCGTACCGGAGCCAGCCA
TTACTACCCTAATACCGCGATCGACTTCCGATATGGGATTCTTCTCCACCGCA
AGGGCGACCGGTTCAGAAACTCTATCGGTACCCGTACAGGAAACGGATCGTA
CGCTTTCGACAACTCCTTTAACCCTTCCGCTGACGCCTGGTGAATCAGAAAAT
ACGCTATTTCCTACGACCGCGCCGGGGATTTCTACCGAGACCCCGAGCGCGG
CACATGAAACTACACAAACCCAAAGTGCAGAAACGGTCGTCTTTACGCAAAG
TCCGAGTACCGAGTCGGAAACCGCGCGATCCCAGTCGCAAGAACCGTGGTAT
TTTACTCAAACTCCTTCGACTGAACAAGCGGCACTTACTCAGACGCAAATAGC
AGAAACGGAGGCGTTGTTTACTCAGACTCCGAGTGCGGAACAAATGACTTTT
ACTCAGACTCCGGGTGCAGAAACCGAAGCGCCTGCCCAGACCCCGTCGACGA
TACCCGAAATATTTACTCAGTCTCGTTCGACGCCCCCCGAAACGGCTCGCGCG
CCTAGCGCGGCGCCGGAAGTCTTTACACAGAGTTCGAGTACCGTAACCGAGG
TGTTTACTCAGACCCCTAGCACGGTACCGAAAACTACTCTAAGTTCGAGTACT
GAACCGGCGATATTTACTCGGACTCAGTCGGCCGGAACTGAAGCCTTTACTC
AGACTTCGAGTGCCGAACCGGACACGATGCGAACTCAGAGTACTGAAACGCA
TTTTTTCACGCAAGCCCCATCGACGGTACCGAAAGCGACTCAGACTCCGAGT
ACAGAGCCGGAGGTCCTAACTCAGAGTCCAAGTACCGAACCTGTACCGTTTA
CCCGGACGCTAGGCGCCGAGCCGGAAATTACTCAGACCCCGAGCGCGGCACC
GGAGGTTTATACTCGATCGTCGAGTACGATGCCAGAAACTGCGCAATCGACA
CCGTTAGCGTCGCAAAATCCGACTTCGTCGGGTACCGGTACGCATAATACTG
AACCGAGGACTTATCCGGTGCAAACGACTCCACATACCCAAAAGCTATACAC
AGAAAATAAGACGCTATCGTTTCCGACGGTTGTATCGGAATTTCACGAAATG
TCGACGGCAGAATCGCAGACGCCGTTACTCGACGTAAAGATAGTCGAGGTTA
AGTTTTCGAACGATGGTGAGGTAACGGCGACATGCGTTTCGACCGTTAAATC
GCCCTATAGGGTCGAAACTAATTGGAAAGTAGACCTAGTCGATGTAATGGAC
GAAATATCTGGTAATAGTCCAGCGGGCGTTTTCAATAGTAACGAAAAGTGGC
AAAAACAGCTATATTACAGAGTAACGGATGGTCGTACGTCGGTACAATTGAT
GTGTCTATCGTGTACGAGCCATTCTCCGGAACCGTACTGTCTATTCGACACGT
CGCTAATAGCGAGGGAAAAAGACATAGCGCCCGAACTATACTTTACGTCTGA
TCCGCAAACCGCATATTGTACGATAACTCTGCCGTCCGGAGTCGTTCCGAGAT
TCGAATGGAGCCTTAATAATGTTTCACTGCCGGAATATTTGACGGCCACGACC
GTTGTTTCGCATACAGCTGGCCAAAGTACAGTGTGGAAGAGCAGCGCGAGAG
CAGGCGAGGCGTGGATTTCTGGCCGGGGAGGCAATATATACGAATGCACCGT
CCTCATCTCAGACGGCACTCGCGTTACTACGCGAAAGGAGAGGTGCTTAACA
AACACATGGATTGCGGTGGAAAACGGTGCTGCTCAGGCGCAGCTGTATTCAC
TCTTTTCTGGACTTGTGTCAGGATTATGCGGGAGCATATCTGCTTTGTACGCA
ACGCTATGGACCGCCATTTATTTTTGA.

### I. Vaccines and Immunogenic Compositions

The present invention relates to the use of the deoptimized rLT/F, the nucleic acid molecules used to construct the recombinant laryngotracheitis virus carrying the fusion gene of Newcastle disease virus, or the host cells to grow them, or any combination thereof, all according to the present invention for the manufacture of a vaccine for poultry. Accordingly, the present invention provides vaccines and/or immunogenic compositions that include a deoptimized rLT/F of the present invention. Such vaccines can be used to aid in the prevention and/or prevent Newcastle disease, and/or maladies associated with ILTV infections. A vaccine according to the present invention can be used for prophylactic and/or for therapeutic treatment, and thus can interfere with the establishment and/or with the progression of an infection and/or its clinical symptoms of disease.

A deoptimized rLT/F of the present invention can be grown by any number of means currently practiced in the field. For example, a recombinant laryngotracheitis virus carrying the fusion gene of Newcastle disease virus of the present invention can be grown through the use of in vitro cultures of primary chicken cells, see e.g., the Examples below where chicken embryo fibroblast cells (CEFs) were used. The CEFs can be prepared by trypsinization of chicken embryos. The CEFs also can be plated in monolayers and then infected with the deoptimized rLT/F. This particular process can be readily scaled up to industrial-sized production.

Therefore, a further aspect of the invention relates to a method for the preparation of the vaccine according to the invention comprising the steps of infecting host cells with deoptimized rLT/F of the present invention, harvestingthe infected host cells, and then admixing the harvested infected host cells with a pharmaceutically acceptable carrier. Suitable methods for infection, culture and harvesting are well known in the art and are described and exemplified herein.

Typically, the infected host cells are harvested while still intact to obtain the deoptimized rLT/F in its cell-associated form. These cells can be taken up in an appropriate carrier composition to provide stabilization for storage and freezing. The infected cells can be filled into glass ampoules, which are sealed, frozen and stored in liquid nitrogen. Accordingly, in certain embodiments of the present invention, the vaccines and/or immunogenic compositions of the present invention are stored frozen and accordingly, comprise a cryropreservative, suchas dimethyl sulfoxide (DMSO), to preserve the frozen infected cells.

Alternatively, when the deoptimized rLT/F is a recombinant HVT, it can be isolated from its host cell, for instance through sonication at the end of culturing, and then taken up into a stabilizer, and freeze-dried (lyophilized) for stable storage or otherwise reduced in liquid volume, for storage, and then reconstituted in a liquid diluent before or at the time of administration. Such reconstitution may be achieved using, for example, vaccine-grade water. In certain embodiments, a lyophilized portion of a multivalent vaccine can comprise one or more antigens and the diluent can comprise one or more other antigens.

In particular embodiments a vaccine of the present invention (or a portion thereof) can be in a freeze-dried form, e.g., as tablets and/or spheres that are produced by a method described in WO 2010/125084, hereby incorporated by reference in its entirety. In particular, reference is made to the examples, from page 15, line 28 to page 27, line 9 of WO 2010/125084, describing a method to produce such fast disintegrating tablets/spheres. Such freeze-dried forms can be readily dissolved in a diluent, to enable systemic administration of the vaccine.

Vaccines and immunogenic compositions can, but do not necessarily include, physiologically compatible buffers and saline and the like, as well as pharmaceutically acceptable adjuvants. Adjuvants can be useful for improving the immune response and/or increasing the stability of vaccine preparations.

Adjuvants are typically described as non-specific stimulators of the immune system, but also can be useful for targeting specific arms of the immune system. One or more compounds which have this activity may be added to the vaccine. Therefore, particular vaccines of the present invention can further comprise an adjuvant. Examples of chemical compounds that can be used as adjuvants include, but are not limited to aluminum compounds (e.g., aluminum hydroxide), metabolizable and non-metabolizable oils, mineral oils including mannide oleate derivatives in mineral oil solution (e.g., MONTANIDE ISA 70 from Seppic SA, France), and light mineral oils such as DRAKEOL 6VR, block polymers, ISCOM's (immune stimulating complexes), vitamins and minerals (including but not limited to: vitamin E, vitamin A, selenium, and vitamin B12) and CARBOPOL®. Other suitable adjuvants, which sometimes have been referred to as immune stimulants, include, but are not limited to: cytokines, growth factors, chemokines, supernatants from cell cultures of lymphocytes, monocytes, cells from lymphoid organs, cell preparations and/or extracts from plants, bacteria or parasites (Staphylococcus aureus or lipopolysaccharide preparations) or mitogens. Generally, an adjuvant is administered at the same time as an antigen of the present invention. However, adjuvants can also or alternatively be administered within a two-week period prior to the vaccination, and/or for a period of time after vaccination, i.e., so long as the antigen, e.g., a deoptimized rLT/F of the present invention persists in the tissues.

The vaccines and/or immunogenic compositions of the present invention may be administered by any route such as in ovo, by parenteral administration, including intramuscular injection, subcutaneous injection, intravenous injection, intradermal injection, by scarification, by oral administration, or by any combination thereof. Furthermore, the deoptimized rLT/F of the present invention can be used and/or combined with additional NDV and/or ILTV, antigens to improve and expand the immunogenicity provided, and/or antigens for other pathogens in order to provide immune protection against such other pathogens. These additional antigens can be either live or killed whole microorganisms, other recombinant vectors, cell homogenates, extracts, proteins, or any other such derivative, provided that they do not negatively interfere with the safety, stability, and efficacy of the vaccine according to the present invention.

The combination of a deoptimized rLT/F of the present invention with an additional NDV, and/or ILTV antigen can be advantageous in those cases in which very virulent field strains of NDV, or ILTV are prevalent, e.g., in a particular geographic region. In this regard, the combination of a deoptimized rLT/F of the present invention with an MDV1, MDV2, or HVT includes the Rispens (MDV1) strain, the SB1 (MDV2) strain, the FC-126 (HVT) strain and/or PB1 (HVT) strain. To improve the response against NDV, deoptimized rLT/F may be combined with an NDV vaccine strain, such as the mild live NDV vaccine strain C2.

Examples of other microorganisms that can be used as antigens together with the deoptimized rLT/F of the present invention include: (i) viruses such as infectious bronchitis virus, adenovirus, egg drop syndrome virus, infectious bursal disease virus, chicken anaemia virus, avian encephalo-myelitis virus, fowl pox virus, turkey rhinotracheitis virus, duck plague virus (duck viral enteritis), pigeon pox virus, avian leucosis virus, avian pneumovirus, and reovirus, (ii) bacteria, such as Escherichia coli, Salmonella spec., Ornitobacterium rhinotracheale, Haemophilis paragallinarum, Pasteurella multocida, Erysipelothrixrhusiopathiae, Erysipelas spec., Mycoplasma spec., and Clostridium spec., (iii) parasites such as Eimeria spec., and (iv) fungi, such as Aspergillus spec. In particular embodiments of the present invention, a deoptimized rLT/F of the present invention can be combined with an infectious bronchitis vaccine.

The combination vaccine can be made in a variety of ways including by combining the deoptimized rLT/F of the present invention with preparations of virus, or bacteria, or fungi, or parasites, or host cells, or a mixture of any and/or all of these. In particular embodiments, the components for such a combination vaccine are conveniently produced separately and then combined and filled into the same vaccine container.

As described above, a vaccine according to the invention can be used advantageously to provide safe and effective immune protection in poultry to multiple diseases, by a single inoculation at very young age or in ovo. Alternatively, as would be apparent to anyone skilled in the art of poultry vaccines the combinations described above also could include vaccination schedules in which the deoptimized rLT/F of the present invention and the additional antigen are not applied simultaneously; e.g., the deoptimized rLT/F may be applied in ovo, and the NDV C2 and/or the IBDV strain (e.g., 89/03) could be applied at a subsequent time/date.

Accordingly, the vaccines of the present invention can be administered to the avian subject in a single dose or in multiple doses. For example, a vaccine of the present invention may be applied at the day of hatch and/or in ovo at day 16-18 (Embryonation Day) ED. When multiple doses are administered, they may be given either at the same time or sequentially, in a manner and time compatible with the formulation of the vaccine, and in such an amount as will be immunologically effective. Therefore, a vaccine of the present invention may effectively serve as a priming vaccination, which later can be followed and amplified by a booster vaccination of the identical vaccine, or with a different vaccine preparation e.g., a classical inactivated, adjuvanted whole-virus vaccine. The volume per dose of a vaccine of the present invention can be optimized according to the intended route of application: in ovo inoculation is commonly applied with a volume between 0.05 and 0.5 ml/egg, and parenteral injection is commonly done with a volume between 0.1 and 1 ml/avian. In any case, optimization of the vaccine dose volume is well within the capabilities of the skilled artisan.

Accordingly, the vaccines of the present invention can be administered to the avian subject in combination with other poultry vaccines. The vaccine of the present invention can be administered with vaccines against diseases including but not limited to Marek's disease virus (MDV), infectiousness Fahrenheit bursal disease virus (IBDV), Newcastle disease virus (NDV), infectious bronchitis virus (IBV), infectious laryngotracheitis virus (ILTV), avian encephalomyclitis virus (AEV), chicken anaemia virus (CAV), bird pox virus (FPV), bird flu virus (AIV), reason virus difficult to understand (reovirus), avian leukosis virus (ALV), avian reticuloendotheliosis virus (REV), avian paramyxovirus (APV), duck hepatitis virus (DHV), aviadenovirus and hemorrhagic enteritis virus (HEV) and/or coccidiosis.

### EXAMPLES

### Example 1: LT LMH Adapted Strain Efficacy and Virulence

### Materials and Methods

### Adaptation of a Contemporary LT Strain to LMH Cells

An LT field virus was isolated and found to be more pathogenic than the current USDA LT strain. In an effort to enable propagation of this virus in cell culture, the strain was passaged in LMH cells.

### Efficacy Studies.

Briefly, 3-week-old specific pathogen free (SPF) chickens were vaccinated by eye-drop, the gold standard for administration, held for 14 days for immunity to develop and observed daily for post vaccination reactions. At the end of the 14-day period, blood and oropharyngeal swabs were collected for serology and detection of vaccine virus, respectively and then chickens were challenged by the infraorbital route with a reference LT challenge strain. Chickens were observed daily for 10 days for clinical signs of LT including increased nasal discharge, moderate to severe conjunctivitis, moist rales, marked dyspnea and expectoration of blood-stained mucus (Garcia, 2013). In addition, chickens were scored 0 to 3 for nasal exudate between 3 and 7-days post challenge. On the days 3, 5, 7, and 9 post challenge, oropharyngeal swabs were collected to evaluate challenge virus load and on day 10 post challenge, tracheas were collected for histopathology.

For a successful outcome, at least 80% of the chickens in the positive control group must develop clinical signs of LT while 90% of the chickens in a vaccine group are free from LT clinical signs.

### Virulence Studies.

The LT LMH adapted, strain were inoculated by the infraorbital route to daily for 10 days for clinical signs of LT as listed above. In addition, chickens were scored from 0 to 3 for nasal exudate between 3 and 7-days post challenge. On the days 3, 5, 7, and 9 post challenge, oropharyngeal swabs were collected to evaluate viral load. At 10 days post challenge, chickens were bled and tracheas collected for histopathology.

### Results

These results were compared to the non-vaccinated, challenged positive control group from the efficacy portion of the trial following LT challenge with a reference LT challenge strain.

LMH-adapted ILTV successfully infected and immunized the chickens. LMH-adapted ILTV has shown to provide adequate protection against USDA ILTV challenge virus. LMH-adapted ILTV is pathogenic; however, the level of pathogenicity and how it compares to the parent virus was not adequately assessed. LMH-adapted virus was titrated in LMH cells while the parent strain was titrated in SPF embryos, therefore the comparison is TCID50 vs EID50. Also, LMH-adapted virus replicates at a low rate making the volume of inoculum high (400 uL to achieve 103 TCID50). It is recommended that the LMH-adapted virus be propagated and titrated in SPF embryos and pathogenicity comparison repeated. The next step is to perform the enzyme-Linked Immunosorbent Assay (ELISA).

TABLE 1
Study design for LT efficacy and virulence of an LMH adaptedstrain.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | 3 | placebo | eye | 10 |
| | | challenged positive controls | week | | drop | |
| 2 | test | LT¹ LMH adapted strain | | 2.0-3.0 | | 10 |
| | efficacy | | | TCID50² | | |

| | | | | |
|---|---|---|---|---|
| 3 | test | LT LMH adapted strain | NA | 10 |
| | virulence | | | |

LT¹ = laryngotracheitis
TCID50² = tissue culture infectious dose in 50%

TABLE 2
Study design for LT efficacy and virulence of an LMH adaptedstrain.

| | | | | | | | Challenge |
|---|---|---|---|---|---|---|---|
| | Treatment | # | | Observe/ | | | (6 wk, 3 |
| Isolator | Group | Chickens | Vaccination | Score | Swab | Bleed | WPV⁴) |
| 1 | Unvaccinated/ | 12 | None | N/A | 28 d | 6 wk | USDA ILTV¹ |
| | Challenged | | | | | | 10^{3.0} EID₅₀² |
| 2 | Unvaccinated/ | 12 | None | N/A | N/A | 6 wk | Parent ILTV |
| | Challenged | | | | | | 10^{3.0} EID₅₀ |
| 3 | Unvaccinated/ | 12 | None | N/A | N/A | 6 wk | LMH⁵- |
| | Challenged | | | | | | adapted ILTV |
| | | | | | | | 10^{3.0} TCID₅₀ |
| 4 | Vaccinated/ | 12 | LMH- | 3-10 | 7 dpv | 21 dpv | USDA ILTV |
| | Challenged | | adapted | dpv³ | (28 d) | (6 wk) | 10^{3.0} EID₅₀ |
| | | | ILTV | | | | |
| | | | 10¹ TCID₅₀ | | | | |
| 5 | Vaccinated/ | 12 | LMH- | 3-10 | 7 dpv | 21 dpv | USDA ILTV |
| | Challenged | | adapted | dpv | (28 d) | (6 wk) | 10^{3.0} EID₅₀ |
| | | | ILTV | | | | |
| | | | 10² TCID₅₀ | | | | |
| 6 | Vaccinated/ | 12 | LMH- | 3-10 | 7 dpv | 21 dpv | USDA ILTV |
| | Challenged | | adapted | dpv | (28 d) | (6 wk) | 10^{3.0} EID₅₀ |
| | | | ILTV | | | | |
| | | | 10³ TCID₅₀ | | | | |
| Total | | 72 | | 36 | 48 | 72 | |
| Chickens | | | | | | | |

ILTV¹ = infectious laryngotracheitis virus
TCID50² = tissue culture infectious dose in 50%
DPV³ = day(s) post-vaccination
WPV⁴ = week(s) post-vaccination
LMH⁵ = Leghorn male hepatoma cell line

TABLE 3
Detection of ILTV in oropharyngeal swabbings via qPCR
and summary of gross lesion development post-vaccination
with different amounts of LMH-adapted ILTV

| | ILTV1 qPCR | Gross Lesions |
|---|---|---|
| Treatment Group | Pos/Tot³ | Pos/Tot |
| Unvaccinated | 0/12 | 0/12 |
| 10¹ TCID₅₀ | 6/12 | 1/12 |
| LMH-adapted ILTV | | |
| 10² TCID₅₀² | 10/12 | 2/12 |
| LMH-adapted ILTV | | |
| 10³ TCID₅₀ | 7/12 | 2/12 |
| LMH-adapted ILTV | | |

ILTV¹ = infectious laryngotracheitis virus
TCID50² = tissue culture infectious dose in 50%
Pos/Tot³ = ratio of positive results to total observations

TABLE 4
Pathogenicity assessment of three ILTV strains and protection against
USDA ILTV strain provided by vaccination with LMH-adapted ILTV.

| | | ILTV¹ | % | | Gross | | |
|---|---|---|---|---|---|---|---|
| | Challenge | qPCR | PCR | | Lesions | % | % |
| Group | Strain | Pos/Tot | Positive | 2^{-ΔΔCta} | Pos/Tot³ | Susceptible | Protected |
| Unvaccinated | USDA | 11/12 | 92% | 440.5 | 11/12 | 92% | 8% |
| | Parent | 12/12 | 100% | 2,853.6 | 11/12 | 92% | 8% |
| | LMH- | 11/12 | 92% | 48.6 | 11/12 | 92% | 8% |
| | Adapted | | | | | | |
| 10¹ TCID₅₀² | USDA | 5/12 | 42% | 8.8 | 3/12 | 25% | 75% |
| LMH-adapted | USDA | 3/12 | 25% | 1.5 | 0/12 | 0% | 100% |
| ILTV | USDA | 3/12 | 25% | 1.0 | 0/12 | 0% | 100% |

ILTV¹ = infectious laryngotracheitis virus
TCID50² = tissue culture infectious dose in 50%
Pos/Tot³ = ratio of positive results to total observations.

### Example 2: Insertion of the Fusion Gene into the LT Genome

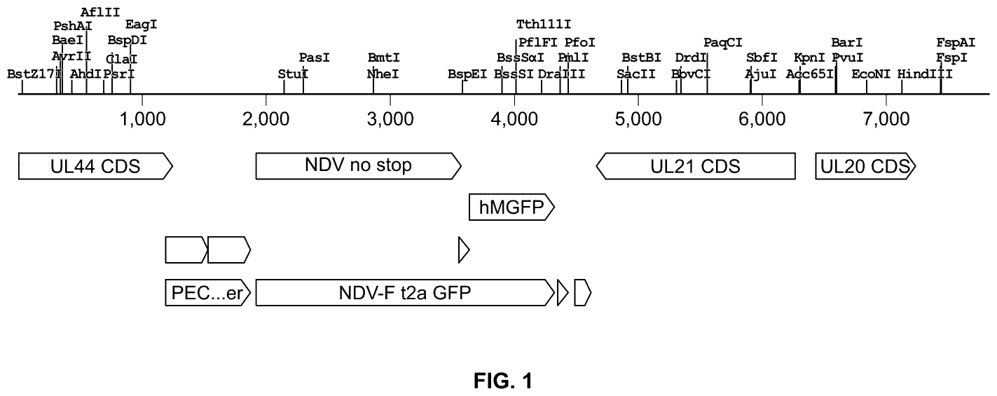

CRISPR/Cas9 editing technologies were used to generate rLT/F. Briefly, LMH cells were transfected with Ribonucleoprotein Complexes (RNPs) Cas9/Cpf1 with complexed guide as well as repair templates containing a promoter, NDV fusion gene, and selection cassette. The guides were directed to cut at PAM sites in the targeted insertion sites. The sites targeted for insertion include UL45/46, sORF-1, UL44/21 and UL3/4. Guides were selected based on guide cutting efficiency assays to detect the presence of non-homologous end joining (NHEJ). DNA isolated from transfected/infected cells were subjected to amplicon sequencing as well as in vitro RNP digestion of amplicons and visualized on a standard agarose gel. Specifically, LMH cells were transfected with Cas9 mRNA and guide complex (TR) or untransfected (WT) and then subsequently infected with LT 4 hours post transfection. Genomic DNA was harvested 3 days post infection. The UL45/46 region was amplified as a 524 bp product (FIG. 1). The guide 2 directs cutting at the 3′ end of the UL45 CDS. An RNP with Cas9 protein and guide 2 and used to assess cutting of the amplicon from WT and TR LT populations. Reduced cutting is easily detected in the TR population vs the WT population indicating efficient cutting at the target site.

The antigen cassette also carries a green fluorescent protein (GFP) gene for identification of recombinants (FIG. 1). These components were introduced by nucleofection into an LMH adapted LT strain and selected based on PCR positivity, antigen expression as well as GFP fluorescence.

Once a candidate was identified, the selection cassette was removed using CRISPR/Cas targeted to a duplicated scrambled (designated sgA) PAM sequence positioned before and after the selection cassette. The sgA is PAM sequence that is not present in the LT genome.

### Example 3: Evaluation of In Vitro Fusion Gene Expression and Genetic Stability of rLT/F

Expression levels of the fusion gene in rLT/F candidates with different insertion sites were evaluated by in vitro methods and those with highest relative expression were selected. To determine the genetic stability, the insert site were sequenced to verify the inserted DNA before and after 10 passages in LHM cells. Stable rLT/F candidates were selected for in vivo New Castle Disease (ND) efficacy.

### Example 4: ND Efficacy to Evaluate Insertion Sites of rLT/F Candidates

To determine the expression of the fusion gene from various insertion sites in the LT genome. Briefly, 3-week-old SPF chickens were vaccinated by eye-drop, held for 20 days for immunity to develop and observed daily for post vaccination reactions. At the end of the 20-day period, blood and oropharyngeal swabs were collected for serology and detection of vaccine virus, respectively and then chickens were challenged with a neurotrophic NDV by the intramuscular route. Chickens were observed daily for 14 days for neurological signs such as head or muscle tremors, torticollis, paralysis of one wing or one leg and mortality (Miller, 2013). Successful outcomes were determined by comparing the efficacy of the various rLT/F candidates with the fusion gene inserted into different insertion sites.

TABLE 4
Study design for ND efficacy to determine
the best insertion site into the LT genome.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | 3 | placebo | eye | 5 |
| | | non-challenged | week | | drop | |
| | | negative controls | | | | |
| 2 | | Placebo-vaccinated, | | | | 10 |
| | | challenged positive | | | | |
| | | controls | | | | |
| 3 | test | rLT/F¹ candidate 1 | | 2.0-3.0 | | 10 |
| 4 | | rLT/F candidate 2 | | TCID₅₀² | | 10 |
| 5 | | rLT/F candidate 3 | | | | 10 |

rLT/F¹ = F gene of Newcastle disease virus inserted into the laryngotracheitis virus genome;
TCID₅₀² = tissue culture infectious dose in 50%

### Example 5: ND Efficacy in Maternal Antibody Positive Chicks

To verify that maternal antibodies specific to ND do not interfere with day-old vaccination, efficacy wereevaluated by vaccinating layer or broiler chickens at day-old. Briefly, to prove that maternal antibodies to NDV are present, several day-old chickens werebled and serology conducted. Other day-old chickens will be vaccinated by eye-drop, and held for 6 to 7 weeks or until the maternal antibody decreases. At the end of the 6 to 7-week period, oropharyngeal swabs werecollected for detection of vaccine virus, and then chickens werechallenged with a neurotrophic NDV by the intramuscular route. Chickens wereobserved daily for 14 days for neurological signs of ND as described above. Successful outcomes weredetermined by comparing the results of rLT/F candidates with a rHVT/ND

TABLE 5
Study design for ND efficacy in maternal antibody positive chickens.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | Day | placebo | eye | 10 |
| | | non-challenged | old | | drop | |
| | | negative controls | | | | |
| 2 | | Placebo-vaccinated, | | | | 20 |
| | | challenged positive | | | | |
| | | controls | | | | |
| 3 | | rHVT/ND¹ | | 3000 pfu³ | | 20 |
| 4 | test | rLT/F² candidate 1 | | 2.0-3.0 | | 20 |
| 5 | | rLT/F candidate 2 | | TCID₅₀⁴ | | 20 |

rHVT/ND¹ = F gene of Newcastle disease virus inserted into the herpesvirus of turkey genome;
rLT/F² = F gene of Newcastle disease virus inserted into the laryngotracheitis virus genome;
pfu³ = plaque forming units;
TCID₅₀⁴ = tissue culture infectious dose in 50%

### Example 6: Identification of Genetic Loci and Designs for Deoptimization of Codon Pairs of LT

In one example a commercial partner was to deoptimize the codon pairs using in silico identification of genetic loci that, based on their proprietary Platform Technology. For example, based on an in silico analysis, about three to four open reading frames (ORFs) will initially be proposed as targets for initial, fully deoptimized sequences (not including the sequences near the flanking regions) within the LT genome, primarily starting with the major surface glycoprotein. Additionally, at least one ORF with a “scrambled” codon sequence design will be designed to resemble the wild-type virus as a positive control for rLT virus recovery. Each design wasproposed as a single rLT/F recombinant with one deoptimized coding sequence within distinct open reading frames.

TABLE 6
LT open reading frame targets for deoptimization.

| | | # | |
|---|---|---|---|
| | ORF | Amino | |
| ORF (nucleotide position) | size | acids | Protein name |
| UL47 126616-128484 also | −1868 | −623 | Tegument (tegument protein |
| sORF1 | | | VP13/14) |
| UL27 41747-39099 | 2648 | 883 | Glycoprotein B |
| US5 129739-132693 | −2954 | −985 | Glycoprotein J abundant on |
| | | | envelope |
| UL44 75683-76924 | −1241 | −414 | Glycoprotein C abundant on |
| | | | envelope |
| UL30 47271-50291 | −3020 | −1007 | DNA polymerase |
| UL42 72398-73693 | −1295 | −432 | Processivity factor for DNA |
| | | | polymerase |
| UL29 47094-44098 | 2996 | 999 | Major single-strand DNA binding |
| | | | protein |
| UL17 86355-88505 | −2150 | −717 | Tegument protein |
| UL25 35392-37107 | −1715 | −572 | DNA packaging protein |
| UL19 79664-83872 | −4208 | −1403 | Major capsid protein |
| UL37 65881-63212 | 2669 | 890 | Tegument protein (tegument |
| | | | protein UL37) |
| US6 132441-133805 | −1364 | −455 | Glycoprotein D (envelope |
| | | | glycoprotein D) |
| US7 133916-135001 | −1085 | −362 | Glycoprotein I (envelope |
| | | | glycoprotein I) |
| UL21 78611-77016 | 1595 | 532 | Nucleocapsid protein (tegument |
| | | | protein UL21) |
| UL49.5 19336-19686 | −350 | −117 | Glycoprotein N Putative viral |
| | | | membrane protein |
| UL32 53233-51479 | 1754 | 585 | Envelope glycoprotein (DNA |
| | | | packaging protein UL32) |
| ORF A 25275-26405 | −1130 | −377 | Hypothetical protein 40 kd (protein |
| | | | IA) |
| ORF B 26448-27467 | −1019 | −340 | Hypothetical protein 34 kd (protein |
| | | | IB) |
| ORF C 28529-27528 | 1001 | 334 | Hypothetical protein 38 and 30 kd |
| | | | (protein IC) |
| ORF D 28639-29760 | −1121 | −374 | Hypothetical protein 41 kd (protein |
| | | | ID) |
| ORF E 31067-29838 | 1229 | 410 | Hypothetical protein 44 kd (protein |
| | | | IE) |
| UL10 94758-93580 | 1178 | 393 | Glycoprotein M (envelope gpM) |
| UL23 34667-33576 | 1091 | 364 | Thymidine kinase |
| UL50 19182-17935 | 1247 | 416 | Deoxyuridine triphosphatase |
| | | | dUTPase |
| US4 128651-129526 | −875 | −292 | Glycoprotein G (envelope |
| | | | glycoprotein G) |
| UL03 106948-106349 | 599 | 200 | nuclear protein UL3 |
| UL04 105403-105936 | −533 | −178 | nuclear protein UL4 |
| US9 or US8A 136704- | −779 | −260 | membrane protein US8A |
| 137483 | | | |
| ICP4 internal repeat | 4388 | 1463 | ICP4 (Infected-cell polypeptide 4) |
| 118888-114500 | | | |
| ICP4 internal repeat | −4388 | −1463 | ICP4 (Infected-cell polypeptide 4) |
| 142837-147225 | | | |
| UL09 94653-97382 | −2729 | −910 | Ori binding protein (DNA |
| | | | replication origin-binding |
| | | | helicase) |
| UL46 21888-23558 | −1670 | −557 | Tegument phosphoprotein; alpha- |
| | | | TIF modulation |
| UL54 12082-10787 | 1295 | 432 | Posttranslational regulator of gene |
| | | | expression |

### Example 7: Generation of Deoptimized rLT/F Virus Clones with Deoptimized Codon-Pair Sequences

CRISPR/Cas9 editing technologies were used to generate deoptimized rLT/F. Up to 3-4 ORF designs will be constructed and evaluated. Target ORFs were selected based on published data demonstrating that these ORFs are critical for viral proliferation potential. The inventors are employing their proprietary CRISPR technology for targeted replacement of the wild type (WT) ORF with a fluorescent reporter (RFP) linked to various VICOPA deoptimized ORFs. Prescence of RFP positive plaques is indicative that the recombination event was successful. Using this method, The inventorsisolated RFP positive recombinant ILT with 12.5%, 25%, 50%, 75% and 95% deoptimized ORFs at the US5 locus. Subsequently, the RFP reporter was removed using CRISPR and was then used to test US5 deoptimized ILT for attenuation in chickens. This strategy continues to be employed to test other candidate ORFs that is capable of facilitating the attenuation of ILT. LMH cells are for LT replication in culture as well as introduction of deoptimized sequences. Briefly, LMH cells are transfected with RNPs Cas9/Cpf1 with complexed guide as well as repair templates for each of the defined sequences targeted for deoptimization. Deoptimized target ORFs in rLT/F was clonally isolated and verified by sequence analysis.

### Example 8: Evaluation of Genetic Stability and Replication Profiles of the Recombinant Viruses

LMH cells were infected with purified virus stocks and evaluated for in vitro replication by analyzing virus titers at different time points. Virus stocks were also be assessed for genetic stability by sequencing for up to 20 continuous passages.

### Example 9: LT Screening by In Vivo

If attenuation was not discernible using replication in vitro then, candidates were be screened for attenuation in vivo. Multiple candidates were inoculated by the infraorbital route at equivalent doses to determine attenuation. Broilers wereobserved daily for 10 days for clinical signs of LT as described above. In addition, chickens werescored from 0 to 3 for nasal exudate between 3 and 7-days post challenge. On the days 3, 5, 7, and 9 post challenge, oropharyngeal swabs were collected to evaluate viral load. At 10 days post challenge, weights wereobtained and tracheas collected for histopathology. Attenuated vaccine candidates were defined by 1) clinical signs and/or nasal exudate comparable to the tissue culture origin (TCO) vaccine 2) clinical signs and/or nasal exudate less than the parent/reference strain and 3) a significant difference in weights compared to the parent strain but not the TCO vaccine. Depending on the number of candidates screened, multiple studies may be performed. If candidates from different studies are selected, attenuation will be re-evaluated in the same study.

TABLE 7
Study design for LT screening by in vivo attenuation is as follows:

| | | | Vaccination | | | |
|---|---|---|---|---|---|---|
| Group | Group | | | | | # |
| # | purpose | Vaccine name | age | dose (10^{x}) | route | chickens |
| 1 | controls | Parent | 3 week | 3.0-4.0 | infraorbital | 10 |
| 2 | | TCO¹ | | TCID₅₀⁴ | | 10 |
| 3 | test | rLT/F² | | | | 10 |
| | groups | candidate 1 | | | | |
| 4 | | rLT/F | | | | 10 |
| | | candidate 2 | | | | |
| 5 | | rLT/F | | | | 10 |
| | | candidate 3 | | | | |
| 6 | | rLT/F | | | | 10 |
| | | candidate 4 | | | | |
| 7 | | rLT/F | | | | 10 |
| | | candidate 5 | | | | |
| 8 | | rLT/F | | | | 10 |
| | | candidate 6 | | | | |

TCO¹ = tissue culture origin vaccine
rLT/F² = F gene of Newcastle disease virus inserted into the laryngotracheitis virus genome
TCID₅₀³ = tissue culture infectious dose in 50%

### Example 10: LT Preliminary Efficacy and Safety in 3-Week-Old SPF Chickens

To determine the preliminary efficacy and safety of live attenuated LT candidates, the first efficacy study is designed with the inclusion of a safety test in the chickens from same hatch as the efficacy test. If this study is successful, the next step is to evaluate efficacy and safety in day-old SPF chickens and day-old broilers, optimal target age for administration.

### Efficacy Studies.

Briefly, 3-week-old SPF chickens werevaccinated by eye-drop, the gold standard for administration, and the administered highest relevant dose estimated at 103 to 10⁴ TCID₅₀ (tissue culture infectious dose in 50%) to give the vaccine the best chance to succeed. Then chickens wereheld for 14 days for immunity to develop and observed daily for post vaccination reactions. At the end of the 14-day period, blood and oropharyngeal swabs werecollected for serology and detection of vaccine virus, respectively and then chickens were challenged by the infraorbital route. Chickens were observed daily for 10 days for clinical signs of LT as described above. In addition, chickens were scored 0 to 3 for nasal exudate between 3 and 7-days post challenge. On the days 3, 5, 7, and 9 post challenge, oropharyngeal swabs were collected to evaluate challenge virus load and on day 10 post challenge, tracheas were collected for histopathology. For a successful vaccine outcome, at least 80% of the chickens in the positive control group must develop clinical signs of LT while 90% of the chickens in a vaccine group are free from LT clinical signs. In addition, the vaccine candidate should be similar to a control chicken embryo origin (CEO) vaccine by pre-challenge serology and post-challenge by the following, (1) a quantitative scoring method of nasal exudate from 0 to 3, (2) reduction of challenge virus load in the trachea and (3) microscopic tracheal lesions.

### Safety Studies

Briefly, 3-week-old SPF chickens were inoculated intratracheally with the vaccine candidate at the same dose used in the efficacy portion of the trial. Chickens were observed daily for 14 days for mortality. Unsafe vaccine outcomes are defined as, if more than 20 percent of the chickens die during the observation period, the virus is unsatisfactory.

TABLE 8
Study design for preliminary LT efficacy and safety
in 3-week-old SPF chickens is as follows:

| | | | Vaccination | | | |
|---|---|---|---|---|---|---|
| Group | Group | | | | | # |
| # | purpose | Vaccine name | age | dose (10^{x}) | route | chickens |
| 1 | efficacy | Placebo-vaccinated, | 3 | placebo | eye drop | 10 |
| | controls | non-challenged | week | | | |
| | | negative controls | | | | |
| 2 | | Placebo-vaccinated, | | | | 20 |
| | | challenged | | | | |
| | | positive controls | | | | |
| 3 | | CEO¹ | | 3.0 EID₅₀³ | | 20 |
| 4 | efficacy | rLT/F² candidate 1 | | 3.0-4.0 | | 20 |
| 5 | test | rLT/F candidate 2 | | TCID₅₀⁴ | | 20 |
| | groups | | | | | |
| 6 | safety | CEO | 3 | 3.0 EID₅₀ | intratracheal | 25 |
| | controls | | week | | | |
| 7 | safety | rLT/F candidate 1 | | 3.0-4.0 | | 25 |
| 8 | test | rLT/F candidate 2 | | TCID₅₀ | | 25 |
| | groups | | | | | |

CEO¹ = embryo origin vaccine;
rLT/F² = F gene of Newcastle disease virus inserted into the laryngotracheitis virus genome;
EID₅₀³ = embryo infectious dose in 50%;
TCID₅₀⁴ = tissue culture infectious dose in 50%

### Example 11: LT Efficacy and Safety in Day-Old SPF Chickens

Optimally, administration is to day-old chickens by spray at the hatchery verses 3-week-old chickens vaccinated in the field by drinking water. Efficacy was conducted as above except that the vaccine candidates were administered by multiple, lowering doses to day-old SPF chickens. If efficacy is demonstrated in day-old SPF chickens, then safety and efficacy in broilers were evaluated as outlined below. If efficacy is not demonstrated, then studies were conducted in 3-week-old SPF and broiler chickens to verify efficacy and safety by drinking water administration.

TABLE 9
Study design for LT efficacy and safety in day-old SPF chickens.

| | | | Vaccination | | | |
|---|---|---|---|---|---|---|
| Group | Group | | | | | # |
| # | purpose | Vaccine name | age | dose (10^{x}) | route | chickens |
| 1 | efficacy | Placebo-vaccinated, | Day | placebo | eye drop | 10 |
| | controls | non-challenged | old | | | |
| | | negative controls | | | | |
| 2 | | Placebo-vaccinated, | | | | 40 |
| | | challenged positive | | | | |
| | | controls | | | | |
| 3 | | CEO¹ | | 3.0 | | 40 |
| | | | | EID₅₀³ | | |
| 4 | efficacy | rLT/F² candidate 1 | | 3.0-4.0 | | 40 |
| 5 | test | Candidate 2 or | | TCID₅₀⁴ | | 40 |
| | groups | candidate 1 at lower | | | | |
| | | dose | | | | |
| 6 | safety | CEO | Day | 3.0 EID₅₀ | intratracheal | 25 |
| | controls | | old | | | |
| 7 | safety | rLT/F candidate 1 | | 3.0-4.0 | | 25 |
| 8 | test | Candidate 2 or | | TCID₅₀ | | 25 |
| | groups | candidate 1 at lower | | | | |
| | | dose | | | | |

CEO¹ = embryo origin vaccine;
rLT/F² = F gene of Newcastle disease virus inserted into the laryngotracheitis virus genome;
EID₅₀³ = embryo infectious dose in 50%;
TCID₅₀⁴ = tissue culture infectious dose in 50%

### Example 12: LT Safety in Day-Old Broilers

For a thorough proof of concept, we will also evaluate safety in broilers since more post vaccination reactions are observed with the current CEO vaccines in broilers than in layers. In addition, post vaccination reactions were evaluated for vaccine candidates in combination with other normal hatchery vaccinations such as infectious bronchitis (IB) vaccines. Briefly, day-old broilers were vaccinated by eye drop with either vaccine candidates or vaccine candidates in combination with IB vaccine. Comparative groups were vaccinated with a CEO vaccine or CEO vaccine and IB vaccine. Respiratory responses were evaluated daily for 14 days for clinical signs LT and IB including gasping, coughing, sneezing, tracheal rales, and nasal discharge (Jackwood, 2013). On the days 3, 5, 7, and 9 post inoculation, oropharyngeal swabs were collected to evaluate LT and IB load. At 10 days post inoculation, weights were obtained and tracheas collected for histopathology. Unsafe outcomes are defined by 1) dramatically exacerbated respiratory reactions and/or increased LT vaccine load when in combination with IB compared to groups without IB and 2) significant weight loss in groups with IB compared to groups without IB.

TABLE 10
design for LT safety in day-old broilers is as follows:

| | | | Vaccination | | | |
|---|---|---|---|---|---|---|
| Group | Group | | | | | # |
| # | purpose | Vaccine name | age | dose (10^{x}) | route | chickens |
| 1 | control | CEO¹ | Day | 3.0 EID₅₀³ | eye | 25 |
| 2 | test | rLT/F² candidate 1 | old | 3.0-4.0 | drop | 25 |
| 3 | groups | Candidate 2 or | | TCID₅₀⁴ | | 25 |
| | | candidate 1 at | | | | |
| | | lower dose | | | | |
| 4 | control | CEO + IB⁵ vaccine | | 3.0 TCID₅₀ + | | 25 |
| 5 | test | rLT/F candidate 1 + | | label | | 25 |
| | groups | IB vaccine | | | | |
| 6 | | Candidate 2 or | | | | 25 |
| | | candidate 1 at | | | | |
| | | lower dose + IB | | | | |
| | | vaccine | | | | |

CEO¹ = embryo origin vaccine;
rLT/F² = F gene of Newcastle disease virus inserted into the laryngotracheitis virus genome;
EID50³ = embryo infectious dose in 50%;
TCID50⁴ = tissue culture infectious dose in 50%;
IB⁵ = infectiousbronchitis

### Example 13: LT Efficacy in Day-Old Broilers

To verify LT efficacy in a day-old broiler, the study was conducted as above for 3-week-old, SPF chickens except the vaccine candidates were administered by spray to day-old broilers.

TABLE 11
Study design for LT efficacy in day-old broilers is as follows:

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | Day | placebo | spray | 10 |
| | | non-challenged | old | | | |
| | | negative controls | | | | |
| 2 | | Placebo-vaccinated, | | | | 40 |
| | | challenged | | | | |
| | | positive controls | | | | |
| 3 | | CEO¹ | | 3.0 | | 40 |
| | | | | EID₅₀³ | | |
| 4 | test | rLT/F² candidate 1 | | 3.0-4.0 | | 40 |
| 5 | groups | Candidate 2 or | | TCID₅₀⁴ | | 40 |
| | | candidate 1 at | | | | |
| | | lower dose | | | | |

CEO¹ = embryo origin vaccine;
rLT/F² = F gene of Newcastle disease virus inserted into the laryngotracheitis virus genome;
EID50³ = embryo infectious dose in 50%;
TCID50⁴ = tissue culture infectious dose in 50%

### Example 14: LT Efficacy in Maternal Antibody Positive Chicks and Minimum Protective Dose

To verify that maternal antibodies specific to LT do not interfere with day-old vaccination, efficacy was evaluated by vaccinating layer or broiler chickens at day-old. In addition, the minimum protective dose was determined. Efficacy was conducted as above except that the vaccine candidates were administered by multiple, lowering doses to day-old maternal antibody positive chickens instead of day-old SPF chickens. One outcome is that the lowest dose is expected to fail efficacy, verifying the endpoint of the dose response.

TABLE 12
Study design for LT efficacy in maternal antibody
positive chicks andminimum protective dose.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | Day | placebo | spray | 10 |
| | | non-challenged | old | | | |
| | | negative controls | | | | |
| 2 | | Placebo-vaccinated, | | | | 40 |
| | | challenged | | | | |
| | | positive controls | | | | |
| 3 | | CEO¹ | | 3.0 EID₅₀³ | | 40 |
| 4 | test | rLT/F² candidate 1 | | dose 1 | | 40 |
| 5 | groups | rLT/F candidate 1 | | dose 2 | | 40 |
| 6 | | rLT/F candidate 1 | | dose 3 | | 40 |

CEO¹ = embryo origin vaccine;
rLT/F² = F gene of Newcastle disease virus inserted into the laryngotracheitis virus genome;
EID₅₀³ = embryo infectious dose in 50%

### Example 15: ND Efficacy in Maternal Antibody Positive Chicks and Minimum Protective Dose

To verify that maternal antibodies specific to ND do not interfere with day-old vaccination, efficacy was evaluated by vaccinating layer or broiler chickens at day-old. In addition, the minimum protective dose was determined. Efficacy was conducted as above except that the vaccine candidates were administered by multiple, lowering doses. One outcome in addition is that the lowest dose is expected to fail efficacy, verifying the endpoint of the dose response.

TABLE 13
Study design for ND efficacy in maternal antibody
positive chicks and minimum protective dose.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | Day | placebo | spray | 10 |
| | | non-challenged | old | | | |
| | | negative controls | | | | |
| 2 | | Placebo-vaccinated, | | | | 30 to 40 |
| | | challenged | | | | |
| | | positive controls | | | | |
| 3 | | rHVT/ND¹ | | 3000 pfu³ | | 30 to 40 |
| 4 | test | rLT/F² candidate 1 | | dose 1 | | 30 to 40 |
| 5 | | rLT/F candidate 1 | | dose 2 | | 30 to 40 |
| 6 | | rLT/F candidate 1 | | dose 3 | | 30 to 40 |

rHVT/ND¹ = F gene of Newcastle disease virus inserted into the herpesvirus of turkey genome;
rLT/F² = F gene of Newcastle disease virus inserted into the laryngotracheitis virus genome;
pfu³ = plaque forming units

### Example 16: ND Efficacy in Chicken Vaccinated with Experimental Laryngotracheitis Vectored Newcastle Vaccines

To determine if these vaccines are efficacious against Newcastle disease virus (NDV), the vaccinated and controlled groups are challenged with NDV Texas GB and NDV B1 respiratory viruses. Two recombinant LT viral vectors expressing the NDV Fusion protein was evaluated. The F gene was inserted into two different locations on the LT virus genome to evaluate potential differences in efficacy. Each recombinant (rLT/F) has one ND-F gene inserted into the LT virus genome.

The first recombinant LT viral vector (labeled as #1) being evaluation expresses the F protein of NDV with its F gene synthesized from the B1 strain sequence (GenBank: JN863120) and was inserted into the intergenic region between UL44 and UL21. The ILT strain originated from the USDA CEO challenge strain, and it was adapted to grow in the LMH cell line. The second recombinant LT viral vector (labeled as #2) expresses the F protein of NDC with its G gene synthesized from the B1 strain sequence (GenBank: JN863120) and was inserted into the intergenic region between UL3 and UL4. The ILT strain, designated 1847 and characterized as genotype IV, originated from a ILT virulent strain obtained from the University of Georgia, and it was adapted to grow in the LMH cell line.

### Efficacy Studies.

Briefly, 3-week-old specific pathogen free (SPF) chickens were vaccinated by eye-drop, the gold standard for administration. At the end of the 21- to 27-day period, blood and oropharyngeal swabs were collected for serology and detection of vaccine virus, respectively, and then chickens were challenged by the infraorbital route with the NDV around 28 days post-vaccination. For the groups challenged with the Texas GB strain, clinical signs, and mortality was observed for 14 days post-challenge. For groups challenged with the B1 strain, clinical signs were observed for 5 days post-challenge, and then tracheal swabs were collected and inoculated into embryonated chicken embryos for virus isolation.

NDV Texas GB strain was administered at a dose of 104 EID50 by the intramuscular route. NDV B1 strain was administered at a dose of 104.0EID50 by the intraocular route.

The NDV hemagglutination inhibition (HI) assay was used to evaluate the serological response for ND and ELISA was used to evaluate the serological response for LT. A qPCR assay was used to evaluate the presence of LT virus remaining in the trachea prior to challenge.

### Titration of Vaccine.

Following vaccination, titration of the vaccine in LMH cells by 10-fold serial dilution was performed. After 5 days of incubation, monolayers were washed with PBS and fixed for 10 min with 1:1 methanol:acetone mixture. For the immunofluorescence assay, the primary antibody is a monoclonal recognizing the NDV Fusion protein and a secondary antibody is an anti-mouse fluorescent conjugated antibody.

### NDV B1 Challenge and Observations.

For evaluation of efficacy against ND B1 challenge strain, chickens were inoculated by intraocular route at 28 days post vaccination. Chickens were observed daily for 5 days post-challenge for respiratory symptoms (i.e. snicking, rales, nasal discharge). Then the trachea was swabbed for each chicken and inoculated into the allantoic fluid of 10-day-old embryonated chicken eggs. Isolation of the B1 strain was confirmed by embryo mortality with HA activity or HA activity up to 7 days of incubation.

### NDV Texas GB Challenge and Observations.

Per 9CFR, 113.329 (c) (3), efficacy against ND challenge with the Texas GB strain, chickens were challenged by intramuscular route at 28 days post vaccination with at least 104.0 EID50 per chicken. Chickens were observed daily for 14 days post-challenge for neurological signs (i.e. tremors, loss of coordination, paralysis, and death).

### Data Analysis.

To verify protection against NDV B1, efficacy was evaluated by the presence or absence of the B1 strain isolated from the tracheal swabs.

To verify protection against NDV Texas GB, efficacy was evaluated by the presence or absence of neurological clinical signs or mortality due to ND.

TABLE 14
ND Efficacy in Chicken Vaccinated with Experimental Laryngotracheitis
Vectored Newcastle Vaccines (Part 1)

| | | | | | 3 wpv² | | 4 wpv | | |
|---|---|---|---|---|---|---|---|---|---|
| | | Target | Actual | | LT | | bleed | | 4 wpv |
| | | Vaccine | Vaccine | | ELISA | | for LT | | oropharyngeal |
| Grp | Treatment | dose | dose | # | pos/total | | ELISA | | swab for LT |
| # | group | TCID₅₀¹ | TCID₅₀¹ | vaccinated | (GMT) | | (GMT) | | qPCR |
| 1 | Vectormune | label | 3080 | 40 | 0/20 | (5) | 0/20 | (2) | 0/20 (0%) |
| | ND⁷ | | | | | | | | |
| 2 | negative | 0 | 0 | 5 | NA | | NA | | 0/5 (0%) |
| | controls | | | | | | | | |
| 3 | Placebo- | 0 | 0 | 20 | 0/10 | (1.8) | 0/7 | (1) | 0/10 (0%) |
| | vaccinated, | | | | | | | | |
| | ND-challenged | | | | | | | | |
| 4 | rLT/F #2 12 | 10³ | 10^{2.80} | 40 | 4/19 | (114) | 6/19 | (78) | 1/20 (5%) |
| | well | | | | | | | | |
| 5 | rLT/F⁶ #1 | 10³ | 10^{2.83} | 40 | 2/20 | (5) | 3/20 | (4) | 0/20 (0%) |
| | picked | | | | | | | | |

TCID₅₀¹ = tissue culture infectious dose in 50%
WPV² = weeks post vaccination
rLT/F⁶ = F gene of Newcastle disease virus inserted into the laryngotracheitis virus genome
Vectormune ND⁷ = USDA licensed vaccine
SQ⁸ = Subcutaneous
N/A⁹ = not applicable
GMT¹⁰ = Geometric Mean Titer

TABLE 15
ND Efficacy in Chicken Vaccinated with Experimental Laryngotracheitis
Vectored Newcastle Vaccines (Part 2).

| | | | | | | | 4 wpv NDV | | | | |
|---|---|---|---|---|---|---|---|---|---|---|---|
| | | | | | | | Texas GB | | 4wpv NDV B1 Challenge | | |
| | | | | | | | Challenge | | (IO⁵, 10^{3.3}EID50) | | |
| | 3 wpv ND | | 4 wpv ND | | 4wpv ND | | (IM⁴, 10⁴ EID₅₀) | | # Embryo | | |
| Grp | HI³ pos ≥8 | | HI pos ≥8 | | ELISA | | # protected/ | | mortality/ | # HA pos/ | |
| # | (GMT) | | (GMT) | | (GMT) | | total # (%) | | total # (%) | total # (%) | |
| 1 | 37/40 | (19) | 36/40 | (21) | 17/20 | (1849) | 19/19 | (100%) | 7/20 (35%) | 11/20 | (55%) |
| 2 | 0/5 | (2.6) | 0/5 | (1) | NA | | N/A | | N/A | N/A | |
| 3 | 0/20 | (2) | 0/20 | (1) | 0/7 | (1) | 0/10 | (0%) | 5/10 (50%) | 6/10 | (60%) |
| 4 | 22/39 | (6.6) | 1/39 | (3.4) | 2/19 | (184) | 13/20 | (65%) | 8/19 (42%) | 18/19 | (85%) |
| 5 | 21/40 | (6.3) | 3/40 | (2.5) | 2/20 | (53) | 6/20 | (30%) | 7/20 (35%) | 8/20 | (40%) |

TCID₅₀¹ = tissue culture infectious dose in 50%
WPV² = weeks post vaccination
HI³ = hemagglutination inhibition assay
IM⁴ = Intramuscular
IO⁵ = Intraocular
rLT/F⁶ = F gene of Newcastle disease virus inserted into the laryngotracheitis virus genome
Vectormune ND⁷ = USDA licensed vaccine
SQ⁸ = Subcutaneous
N/A⁹ = not applicable
GMT¹⁰ = Geometric Mean Titer

### Example 17: Avian Virus Efficacy to Evaluate Insertion Sites of rLT/F Candidates

To determine the expression of the fusion gene from various insertion sites in the LT genome. Briefly, 3-week-old SPF chickens are vaccinated by eye-drop, held for 20 days for immunity to develop and observed daily for post vaccination reactions. At the end of the 20-day period, blood and oropharyngeal swabs are collected for serology and detection of vaccine virus, respectively and then chickens are challenged with a neurotrophic avian virus by the intramuscular route. Chickens will be observed daily for 14 days for neurological signs such as head or muscle tremors, torticollis, paralysis of one wing or one leg and mortality (Miller, 2013). Successful outcomes will be determined by comparing the efficacy of the various rLT/F candidates with the fusion gene inserted into different insertion sites. Avian virus candidates include infectious bronchitis virus (IBV), infectious laryngotracheitis virus (ILTV) or avian influenza (AI).

TABLE 4
Study design for avian virus efficacy to determine
the best insertion site into the LT genome.

| | | | Vaccination | | | |
|---|---|---|---|---|---|---|
| Group | Group | | | | | # |
| # | purpose | Vaccine name | age | dose (10^{x}) | route | chickens |
| 1 | controls | Placebo-vaccinated, | 3 | placebo | eye | 5 |
| | | non-challenged | week | | drop | |
| | | negative controls | | | | |
| 2 | | Placebo-vaccinated, | | | | 10 |
| | | challenged positive | | | | |
| | | controls | | | | |
| 3 | test | rLT/F¹ candidate 1 | | 2.0-3.0 | | 10 |
| 4 | | rLT/F candidate 2 | | TCID₅₀² | | 10 |
| 5 | | rLT/F candidate 3 | | | | 10 |

rLT/F¹ = F gene of infectious bronchitis virus (IBV), infectious laryngotracheitis virus (ILTV) or avian influenza (AI) inserted into the laryngotracheitis virus genome;
TCID₅₀² = tissue culture infectious dose in 50%

INCORPORATION BY REFERENCE: All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope.

Other aspects, advantages, and modifications are within the scope of the claims set forth below. All publications, patents, and patent applications cited in this specification are herein incorporated by reference as if each individual publication or patent application were specifically and individually indicated to be incorporated by reference.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed:

1. A composition for immunizing an avian subject against a pathogen comprising:
a) a recombinant laryngotracheitis virus vector (rLT); and
b) a fusion gene of an avian virus (F) inserted into the recombinant laryngotracheitis virus vector (rLT/F);
wherein the recombinant laryngotracheitis virus vector inserted with the fusion gene of the avian virus (rLT/F) is deoptimized to generate deoptimized rLT/F vaccine candidates; and
wherein the deoptimized rLT/F vaccine candidates are administered to the subject in need thereof.

2. The composition according to claim 1, wherein the avian virus is selected from Newcastle disease virus (NDV), infectious bronchitis virus (IBV), infectious laryngotracheitis virus (ILTV) or avian influenza (AI).

3. The composition according to claim 1, wherein the avian virus is Newcastle disease virus (NDV), and the composition has at least 99% sequence identity to SEQ ID NO: 1.

4. The composition according to claim 1, wherein the deoptimized vaccine candidates comprise a deoptimized coding sequence having at least 99% sequence identity to SEQ ID NO:22-26.

5. The composition according to claim 1, wherein the deoptimized rLT/F vaccine is administered to the subject to induce an immune response against the pathogen using a gel administration.

6. A deoptimized rLT/F vaccine for immunizing an avian subject in need thereof against a pathogen comprising the deoptimized rLT/F composition according to claim 1, wherein an effective immunizing dose of the deoptimized rLT/F vaccine is administered to the subject to induce an immune response against the pathogen.

7. The deoptimized rLT/F vaccine according to claim 6, wherein the pathogen is selected from Newcastle disease virus (NDV), infectious bronchitis virus (IBV), infectious laryngotracheitis virus (ILTV) or avian influenza (AI).

8. The deoptimized rLT/F vaccine according to 6, wherein the avian virus is selected from Newcastle disease virus (NDV), infectious bronchitis virus (IBV), infectious laryngotracheitis virus (ILTV) or avian influenza (AI).

9. The deoptimized rLT/F according to claim 6, wherein gene targets are fully deoptimized within the LT genome.

10. A method of developing the deoptimized rLT/F vaccine according to claim 6 comprising the steps of:
a. inserting the fusion gene of an avian virus (F) in a recombinant laryngotracheitis vector (rLT);
b. evaluating insertion sites of the avian virus fusion gene in the recombinant laryngotracheitis vector;
c. deoptimizing the recombinant laryngotracheitis vector carrying the fusion gene of the avian virus (rLT/F); and
d. generating deoptimized rLT/F vaccine candidates;
wherein the deoptimized rLT/F vaccine is administered into an avian subject in need thereof.

11. The method according to claim 10, wherein the recombinant laryngotracheitis vector comprises the laryngotracheitis genome.

12. The method according to claim 10, wherein the rLT/F is deoptimized using deoptimization based on codon pair bias.

13. The method according to claim 10, wherein gene editing technologies are used to generate deoptimized rLT/F.

14. The method according to claim 10, wherein the vaccine is administered intratracheally.

15. The method according to claim 10, wherein the vaccine is administered by drinking water.

16. The method according to claim 10, wherein the vaccine is administered by spray.

17. The method according to claim 10, wherein the vaccine is administered by gel droplets or beads.

18. The method according to claim 10, wherein the vaccine is administered in multiple, lowering doses.

19. The method according to claim 10, wherein the vaccine is administered in combination with other vaccinations.

20. The method according to claim 19, wherein the vaccine is administered in combination with an infectious bronchitis (IB) vaccine.
